(19) United States
(12) Patent Application Publication
(10) Pub. No.: US 2025/0157583 A1
(43) Pub. Date: May 15, 2025

(54) MODEL-BASED HOMOGENEITY DETERMINATION OF DATA DETECTION SAMPLE

(71) Applicant: Invivoscribe, Inc., San Diego, CA (US)

(72) Inventors: Junmei Liu, Chandler, AZ (US); Griffin Charles, San Diego, CA (US)

(21) Appl. No.: 18/946,275

(22) Filed: Nov. 13, 2024

Related U.S. Application Data
(60) Provisional application No. 63/548,324, filed on Nov. 13, 2023.

(51) Int. Cl. G16B 40/00

(52) U.S. Cl. CPC G16B 40/00

(57) ABSTRACT

A computing device may receive a detection dataset comprising a set of detection values corresponding to measurements of target entities generated from a sample in a detection analysis. Each detection value in the set corresponds to a measurement of one or more target entities. The computing device may extract one or more features of the detection value in the detection dataset, wherein at least one feature is extracted from a version of a particular detection value. The computing device may input the one or more features of the peaks into a model that is trained based on training samples of past detection datasets. The computing device may generate a computer-automated determination of homogeneity of the target entities in the sample based on an output of the model.

### CROSS-REFERENCE TO RELATED APPLICATION

The present application claims the benefit of U.S. Provisional Patent Application No. 63/548,324, filed on Nov. 13, 2023, which is hereby incorporated by reference in its entirety.

### BACKGROUND

Experimental data, often derived from scientific principles, frequently display patterns that align with established statistical distributions. These distributions are fundamental tools for understanding and describing data variations. Nonetheless, when conducting experiments, a multitude of factors introduce noise and complexities, making it conventionally challenging to ascertain whether a specific dataset genuinely conforms to a specific statistical distribution. This uncertainty has long presented a dilemma in the realm of scientific research that focuses on the accurate modeling and interpretation of experimental data.

In the world of empirical research, noise and various experiment-related factors are ubiquitous challenges that can obfuscate the true nature of data. Factors such as measurement errors, environmental variability, and instrument limitations contribute to deviations from ideal statistical distributions. These deviations can affect conclusions and introduce systematic biases. Consequently, robust methods for assessing the compatibility of experimental data with known statistical distributions have become the focus to extract valuable insights and make informed decisions.

### SUMMARY

Systems and methods are disclosed herein for automatically determining whether a dataset generated from measurements or experiments of samples follows a statistical distribution, such as a Gaussian distribution. By way of example, one or more features may be extracted from the dataset as representative of the data. For example, a feature may be the maximum normalized height of the peaks in the data distribution. The features may be inputted into a model that is trained based on past training data with known results of past measurements or experiments. The model may take the form of a regression model or another machine learning model to generate a score. The score may be compared to a threshold to determine whether the dataset follows the statistical distribution.

In various embodiments, this automated determination process can have broad applications in various technical areas. By way of example, the process may be applied to the result of a signal detection analysis to determine whether the dataset generated from analyzing a sample represents that the sample is homogeneous or not. In some embodiments, a data analytic device receives a detection dataset that includes a set of detection values corresponding to measurements of target entities. The target entities are generated from the sample. Each detection value in the set corresponds to a measure of a target entity. In some embodiments, the data analytic device extracts one or more features of the detection values in the detection dataset to determine whether the dataset follows an expected distribution, such as the Gaussian distribution. A non-homogeneous sample that includes random gene re-arrangement often exhibits a Gaussian distribution of sample target entities. The data analytic device inputs one or more features into a model to make a computer-automated determination of homogeneity of the sample.

### BRIEF DESCRIPTION OF THE DRAWINGS

FIG. 1 illustrates one embodiment of a system environment of a sample analysis system for generating automated determination regarding a detection dataset, in accordance with some embodiments.

FIG. 2 is a conceptual diagram illustrating the target region of an example assay that may be performed in a detection system, in accordance with some embodiments.

FIG. 3 is a conceptual diagram illustrating the target region of an example assay that may be performed in a detection system, in accordance with some embodiments.

FIG. 4A through FIG. 4D illustrates various examples of graphical illustrations of datasets generated from an assay performed by a detection system, in accordance with some embodiments.

FIG. 5 is a flowchart depicting an example process of generating automated determination regarding a detection dataset, in accordance with some embodiments.

FIG. 6A is a flowchart depicting a first example process of generating a computer-automated determination regarding the homogeneity of a detection dataset, in accordance with some embodiments.

FIG. 6B is a flowchart depicting a second example process of generating a computer-automated determination regarding the homogeneity of a detection dataset, in accordance with some embodiments.

FIG. 7 is a conceptual diagram illustrating an example machine learning model, in accordance with some embodiments.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### System Overview

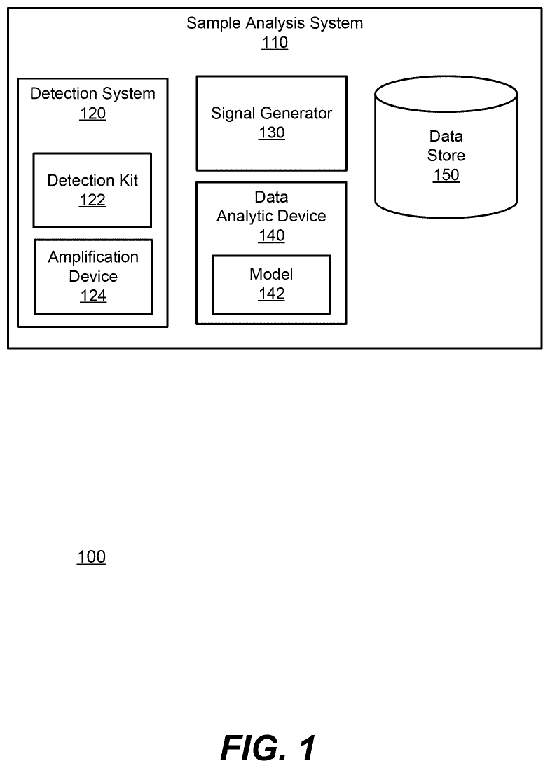

FIG. 1 illustrates one embodiment of a system environment 100 of a sample analysis system 110 for generating automated determination regarding a detection dataset, in accordance with some embodiments. The sample analysis system 110 may include an detection system 120 that includes an detection kit 122 and an amplification device 124, a signal generator 130, a data analytic device 140 that stores a model 142, and a data store 150. In various embodiments, the system environment 100 may include different, fewer, or additional components.

The components in the sample analysis system 110 may each correspond to a separate and independent entity or may be controlled by the same entity. For example, in some embodiments, the detection system 120 may be controlled by one entity that operates a laboratory that analyzes biological samples of subjects. The data analytic device 140 may be controlled by another entity that provides data analysis. In other embodiments, the detection system 120, the signal generator 130, and the data analytic device 140 may be controlled by the same entity. The biological sample is processed in vitro and the data is analyzed in silico in a single setting.

The components in the sample analysis system 110 may be geographically located in the same location or distributed in various locations. In some embodiments, the components in sample analysis system 110 may be geographically located in the same physical housing, consolidating all functionalities into a single analytical device, providing an all-in-one solution for sample analysis. In some embodiments, the components can be distributed across various devices, offering flexibility and scalability. For instance, the detection system 120 is a device that is commercially available, while the data analytic device 140 is a separate device, such as a desktop computer located at a laboratory or facility where the detection system 120 is located. In some embodiments, the data analytic device 140 may also take the form of a remote server, such as a computing server serving as the core of a Software as a Service (SaaS) platform. This remote server allows users to upload the assay data to the computing server to perform further analysis.

While each of the components in the system environment 100 is sometimes described in disclosure in a singular form, the system environment 100 may include one or more of each of the components. For example, different types of detection system 120 may be used to generate assay data for the same data analytic device 140 to analyze.

The detection system 120 includes an detection kit 122 and an amplification device 124. The detection kit 122 is configured to facilitate the preparation and processing of samples for analysis. The detection kit 122 may also be referred to as an assay kit or a test kit. The detection kit 122 is designed to facilitate the preparation, processing, and analysis of biological or chemical samples. In various embodiments, the detection kit 122 typically includes a set of reagents, consumables, and specialized equipment tailored to specific assay requirements. These components may include, but are not limited to, extraction buffers, primers, probes, enzymes, and other materials for sample preparation and reaction initiation. In some embodiments, the detection kit 122 includes primers that may take the form of oligonucleotide primers with nucleotide sequences that are configured to hybridize with nucleotides of the biological sample in target genetic regions. In some embodiments, the primers are fluorescently labeled. The target genetic regions may include conserved genetic regions.

The amplification device 124 allows for the amplification of target molecules, such as nucleic acids. In some embodiments, the amplification device 124 is configured to perform the selective and precise amplification of target molecules within biological or chemical samples. The amplification device 124 may be equipped with components for polymerase chain reaction (PCR) or similar amplification techniques. For example, the 124 may include a thermal cycler for precise temperature control, ensuring denaturation, annealing, and extension steps. In some embodiments, the amplification device 124 accommodates reaction vessels, such as PCR tubes or microplates, where samples are subjected to cycles of heating and cooling to replicate specific DNA or RNA sequences. The replicated nucleotide samples may be referred to as amplicons.

In some embodiments, a signal generator 130 is responsible for generating signals associated with the signal detection analysis. A signal detection analysis may also be referred to as a detection assay such as a biological assay or simply an assay. A detection assay may take the form of an electropherogram, Sanger sequencing, massively parallel sequencing (also referred to as next-generation sequencing, NGS), mass spectrometry, flow cytometry, chromatography, fluorescence in situ hybridization (FISH), polymerase chain reaction (PCR), enzyme-linked immunosorbent assay (ELISA), Southern blotting, or digital droplet PCR. The signals generated by a signal detection analysis (detection assay) may include data from the assay, amplification, or other relevant parameters. In various embodiments, the signals generated by the signal generator 130 may encompass a wide spectrum of data, ranging from the outcomes of the initial assay to the results of the amplification process and various other pertinent parameters. The signal generator 130 may have the capacity to generate and relay signals to generate datasets and provide a comprehensive and real-time understanding of the sample's composition in a wide array of applications, from diagnostics to research.

The data may be referred to as a detection-assay data or a detection-assay data. For simplicity, the data will be referred to as detection-assay data in the remaining of this disclosure but the data may also simply be referred to as detection data.

In some embodiments, an example of a signal generator 130 is a device that generate data signals from a detection assay, such as a sequencing device, a capillary electrophoresis machine, or another suitable machine. Using capillary electrophoresis as an example, an amplicon sample solution may be injected into a capillary tube and injected into a capillary electrophoresis machine. The capillary electrophoresis machine applies electric current to separate the amplicons according to the fragment sizes. An excitation source is used to excite the fluorescent labels of the amplicons. A detector is used to record the intensity of the fluorescent signals. The signal generator 130 may generate a signal chart that includes a set of peaks corresponding to measurements of amplicons that are sorted based on fragment sizes (e.g., numbers of bases in fragments) and intensities. In some embodiments, the signal chart may be stored in the form of a detection dataset.

An example of a detection-assay dataset is an electropherogram dataset, but other forms of data are also possible. Another example of a detection-assay dataset may be a dataset generated by a massively parallel sequencing result. A detection may generate fragments, such as nucleic acid fragments. While nuclei acid fragments are used as the primary example, in various embodiments other fragments may also be analyzed. The type of fragment sizes, as to whether the sizes are base sizes (a number of bases in a fragment), may depend on the type of fragments being analyzed. In some embodiments, the things that are being detected in a detection assay can be referred to as target entities. Target entities can be amplicons, nucleotide sequences (e.g., nucleic acid fragments), etc. A measurement of a target entity can be the sequence of the nucleic acid detected by a sequencing machine, the fragment size measurement in an electropherogram, or another suitable measurement of a target entity in the sample.

In a detection-assay dataset, there can be a set of detection values. Each detection value corresponds to a measurement of one or more target entities. For example, in a sequencing dataset, a measurement of one or more target entities can be a particular sequence read and a detection value corresponding to the sequence read can be the read count of the particular sequence read (e.g., how many counts are sequenced to have the sequence read of AAGTAGTCG). In electropherogram dataset, a measurement of one or more target entities can be a fragment size (a number of bases in a fragment) of one or more amplicons that share the same fragment size. Such individual measurement can be reflected as a peak in an electropherogram chart. A detection value corresponding to a particular fragment size can be the intensity of the peak of the fragment size. In some embodiments, a nuclei-acid measurement may refer to a sequence read in the case of sequencing (e.g., NGS) and a fragment size in the case of electropherogram. These are merely examples of two detection assays. A version of a value may refer to any suitable version of the value that can be used for comparison and analysis. For example, in NGS, a version of a detection value can be the raw read count, an aggregated read count, a normalized read count, a percentage read count, or another statistical measurement of the read count.

A data analytic device 140 is a computing device that may take various forms in different embodiments. In some embodiments, the data analytic device 140 may be the computing component of an all-in-one sample analysis system 110 that includes both the detection system 120 and the data analytic device 140. In some embodiments, the data analytic device 140 is a computing device such as a personal computer (PC), a desktop computer, a laptop computer, a tablet computer, a server-based and/or network linked computer, a smartphone, a wearable electronic device such as a smartwatch, or another suitable electronic device. In some embodiments, the data analytic device 140 may be a server computer that includes one or more processors and memory that stores code instructions that are executed by the one or more processors to perform various processes described herein. In some embodiments, the data analytic device 140 may be a pool of computing devices that may be located at the same geographical location (e.g., a server room) or be distributed geographically (e.g., cloud computing, distributed computing, or in a virtual server network). In some embodiments, the data analytic device 140 may be a collection of servers that independently, cooperatively, and/or distributively provide various products and services described in this disclosure. The data analytic device 140 may also include one or more virtualization instances such as a container, a virtual machine, a virtual private server, a virtual kernel, or another suitable virtualization instance.

In some embodiments, the data analytic device 140 may train one or more models 142 that could include machine learning models, rule-based models, heuristic models, rule-based models, and/or a combination thereof. In some embodiments, a model 142 may be pre-trained and stored in the data analytic device 140 as part of the functionality of the data analytic device 140. In some embodiments, the data analytic device 140 may perform additional training or re-training of the model 142. For example, the data analytic device 140 may train a diagnosis model, an assay analysis model, or any machine learning model. The data analytic device 140 may use machine learning models to perform the functionalities described herein. Example machine learning models include regression models, support vector machines, naïve Bayes, decision trees, k nearest neighbors, random forest, boosting algorithms, k-means, and hierarchical clustering. The machine learning models may also include neural networks, such as perceptrons, multilayer perceptrons, convolutional neural networks, recurrent neural networks, sequence-to-sequence models, generative adversarial networks, or transformers.

In some embodiments, a model 142 includes a set of parameters. A set of parameters for a machine learning model are parameters that the machine learning model uses to process input(s). For example, a set of parameters for a linear regression model may include weights that are applied to each input variable in the linear combination that comprises the linear regression model. Similarly, the set of parameters for a neural network may include weights and biases that are applied to each neuron in the neural network. The data analytic device 140 generates the set of parameters for a machine learning model by training the machine learning model. Once trained, the machine learning model uses the set of parameters to transform inputs into outputs.

In some embodiments, a data store 150 serves as a repository for storing assay data, results, and analysis reports generated by the system. The data store 150 includes one or more storage units such as memory that takes the form of a non-transitory and non-volatile computer storage medium to store various data. The computer-readable storage medium is a medium that does not include a transitory medium such as a propagating signal or a carrier wave. The data store 150 may be used by the data analytic device 140 to store data related to the data analytic device 140. In some embodiments, the data store 150 communicates with other components by a network. This type of data store 150 may be referred to as a cloud storage server. Examples of cloud storage service providers may include AMAZON AWS, DROPBOX, RACKSPACE CLOUD FILES, AZURE, GOOGLE CLOUD STORAGE, etc. In some embodiments, instead of a cloud storage server, the data store 150 is a storage device that is controlled and connected to the data analytic device 140. For example, the data store 150 may take the form of memory (e.g., hard drives, flash memory, discs, ROMs, etc.) used by the data analytic device 140 such as storage devices in a storage server room that is operated by the data analytic device 140.

### Example Assays

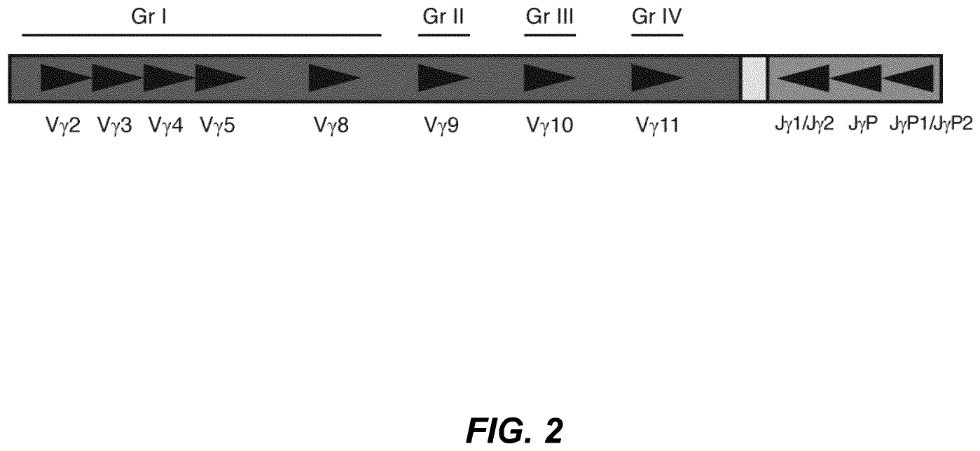

FIG. 2 is a conceptual diagram illustrating the target region of an example assay that may be performed in the detection system 120, in accordance with some embodiments. This example assay may be referred to as the T-Cell Receptor Gamma Gene Rearrangement Assay. The assay is an in vitro diagnostic product intended for PCR-based detection of clonal T-cell receptor gamma chain gene rearrangements in patients with suspect lymphoproliferations. Specifically, the assay can be used to identify clonality in suspect lymphoproliferations.

Rearrangements of the antigen receptor genes occur during ontogeny in B and T lymphocytes. These gene rearrangements generate products that are unique in length and sequence. Polymerase chain reaction (PCR) assays can be used to identify lymphocyte populations derived from a single cell by detecting the unique V-J gene rearrangements present within these antigen receptor loci. In some embodiments, this T-Cell Receptor Gamma Gene Rearrangement Assay employs multiple consensus DNA primers that target conserved genetic regions within the T-cell receptor gamma chain gene. Amplifying the region with fluorescently labeled primers is followed by fractionation by capillary electrophoresis and analysis by instrument software. This DNA-based test is used to detect the vast majority of clonal T-cell populations. The presence or absence of clonality can support the differential diagnosis of reactive lesions and certain T and B cell malignancies.

In some embodiments, the detection kit 122 of the assay includes a single master mix that contains primers that target the Vγ2, 3, 4, 5, 8, 9, 10 & 11 and Jγ1/Jγ2, JγP, and JγP1/JγP2 regions. The PCR amplicons have an expected size range between 159 and 207 base pairs. The detection kit 122 may also include a specimen control size ladder master mix that targets multiple genes and generates a series of amplicons of approximately 100, 200, 300, 400, and 600 base pairs to ensure that the quality and quantity of input DNA is adequate to yield a valid result. In some embodiments, a single thermal cycler program and similar detection methodology are used with the gene clonality assays. This improves consistency and facilitates cross-training on a broad range of our different assays.

In some embodiments, the T-cell receptor Gamma Gene Rearrangement Assay is used for the identification of clonal T-cell populations. FIG. 1 shows the approximate placement of the upstream and downstream DNA primers in the T cell receptor gamma gene. This test amplifies the DNA between primers that target conserved regions within the variable (V) and the joining (J) regions that flank the unique hypervariable antigen-binding region 3 (CDR3). These conserved regions lie on either side of an area within the V-J region where programmed genetic rearrangements occur during the maturation of all B and T lymphocytes. The antigen receptor genes that undergo rearrangement are the immunoglobulin heavy chain and light chains in B-cells, and the T cell receptor genes in T-cells. Each B- and T-cell has a single productive V-J rearrangement that is unique in both length and sequence. Therefore, when DNA from a normal or polyclonal population is amplified using DNA primers that flank the V-J region, a Gaussian distribution (a bell-shaped curve) of amplicon products is produced within an expected size range. This Gaussian distribution reflects the heterogeneous population of V-J rearrangements. In certain cases, where lymphocyte DNA is not present, no product is detected. For DNA from samples containing a clonal population, the yield is one or two prominent amplified products (amplicons) within the valid size range.

Since the antigen receptor genes are polymorphic (including a heterogeneous population of related DNA sequences), it is difficult to employ a single set of DNA primer sequences to target all of the conserved flanking regions around the V-J rearrangement. N-region diversity and somatic mutation further scramble the DNA sequences in these regions. Therefore, a multiplex master mix, which targets multiple V and J regions (FIG. 2), is used to detect the majority of clonal rearrangements. As indicated, clonal rearrangements are identified as one or two prominent, single-sized products within the background of different-sized amplicon products that form the Gaussian distribution around a statistically favored, average-sized rearrangement.

In some embodiments, fluorescence detection is used to resolve the different-sized amplicon products using a capillary electrophoresis instrument. Primers are conjugated with a 6FAM fluorescent dye (fluorophore) so that primers can be detected after excitation by a laser in the capillary electrophoresis instrument. This highly sensitive detection system provides single base pair size resolution and relative quantification. Inter and intra-assay reproducibility in size determination using capillary electrophoresis is approximately 1 to 2 base pairs. This reproducibility and sensitivity coupled with the automatic archiving of specimen data allows for the monitoring, tracking, and comparison of data from individual patients over time.

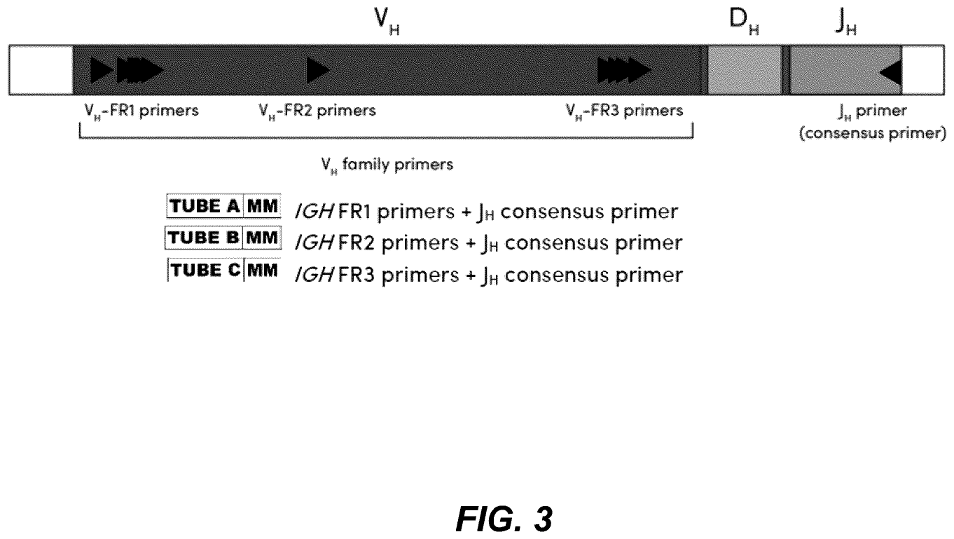

FIG. 3 is a conceptual diagram illustrating the target region of an example assay that may be performed in the detection system 120, in accordance with some embodiments. The assay may be referred to as immunoglobulin heavy chain (IGH) assay, which is an in vitro diagnostic assay intended for capillary electrophoresis-based detection of clonality in immunoglobulin heavy chain gene rearrangements in peripheral blood specimens. In some embodiments, the IGH assay may serve as an adjunctive method for the diagnosis of patients suspected to have a B-cell lymphoproliferative disease.

Rearrangements of the antigen receptor genes occur during ontogeny in B lymphocytes and vary in length, sequence, and function. When polymerase chain reaction (PCR) is applied to these gene rearrangements, products are generated that are unique in length and sequence for each cell. Thus, this methodology can be applied to identify lymphocyte populations derived from a single cell by identifying the unique V-J gene rearrangements present within these antigen receptor loci. The IGH Assay employs multiple consensus DNA primers that target conserved genetic regions within the immunoglobulin heavy chain gene, amplifying the region with fluorescently labeled primers, followed by capillary electrophoresis mediated fractionation and result-determining analysis by assay-specific software. In some embodiments, this DNA-based test is used to detect the vast majority of clonal B-cell populations. The presence or absence of clonality can support the differential diagnosis of reactive lesions and B-cell malignancies.

In some embodiments, the detection kit 122 of the IGH Assay includes three master mixes targeting framework 1, 2, and 3 regions within the variable (VH) region and the joining (JH) region of the immunoglobulin heavy chain locus, as well as a positive (IGH POS [+]) control, a negative (NEG [−]) control, a no template control (NTC), and Taq DNA polymerase. The sample is amplified with each framework (FR)-specific master mix in three PCRs. Results generated by each master mix can be compiled to establish a final clonality result for the sample.

The IGH Assay is designed to target the conserved framework (FR) of the variable (VH) and joining (JH) regions which lie on either side of an area within the VH-JH region where programmed genetic rearrangements occur during the maturation of all B lymphocytes. Because the antigen receptor genes are polymorphic (including a heterogeneous population of related DNA sequences) and N-region diversity and somatic mutation further scramble the DNA sequences in these regions, it is difficult to employ a single set of PCR primers to target all of the conserved regions flanking the VH-JH rearrangement. Therefore, three multiplex master mixes, each targeting one of the three FR regions are used to identify the majority of complete rearrangements. This is illustrated in FIG. 3. Consensus primers are conjugated with fluorescent dyes to enable the detection of the resulting amplicons by a capillary electrophoresis instrument.

In FIG. 3, the organization of a rearranged immunoglobulin heavy chain gene on chromosome 14 is illustrated. The arrows represent the relative positions of primers that target the conserved framework (FR1-3) and the downstream consensus JH gene segments. The amplicon products generated from each of these regions can be differentially detected when fluorescent primer sets are used with capillary electrophoresis instruments that employ differential fluorescence detection.

While two examples of signal detection analysis are illustrated in FIG. 2 and FIG. 3, in various embodiments, the signal detection analysis may take a different from. For instance, the input data may come from various analytical techniques beyond electropherograms, such as datasets generated from Next Generation Sequencing, where the size-based data versus intensity distribution can also be analyzed. Additional examples of signal detection analysis could include gas or liquid chromatography, mass spectrometry, fluorescence intensity readings, and absorbance spectra from UV-Vis spectrophotometry. These examples of signal detection analysis may provide distributions of size or molecular weight against intensity and the automated determination process described in this disclosure may also be applied.

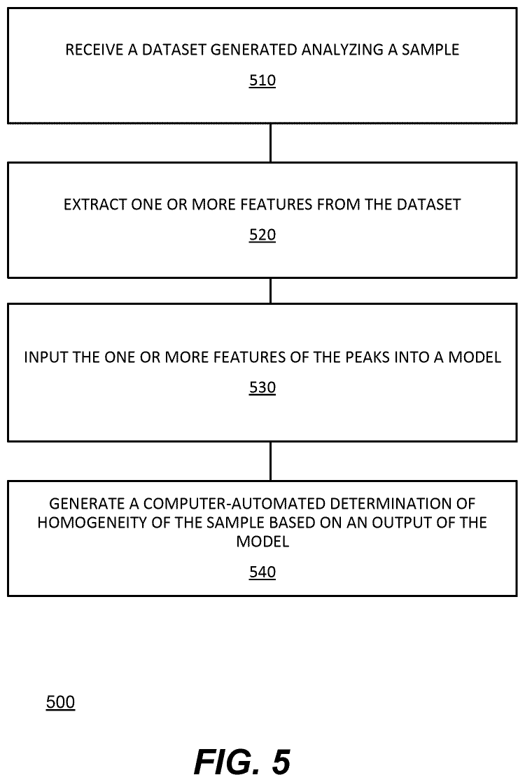

A third example of assay is NGS (not shown in figures). Since NGS data output provided a larger dataset, in some embodiments, data can be grouped by V-J gene type based on information such as aligned sequence length, gene type (e.g., V-gene, J-gene), and percentage of total reads. For each V-J gene type, the sum of the percentage of total reads can be calculated as percentage reads (a version of a detection value), and the mean or median of the aligned sequence length can be calculated as size (another example of a version of a detection value). This yielded percentage read and size values for each V-J gene type, resulting in a data format similar to electropherogram data. The algorithmic method, further discussed in association with FIG. 5) can then applied to these grouped data.

### Example Data Illustration

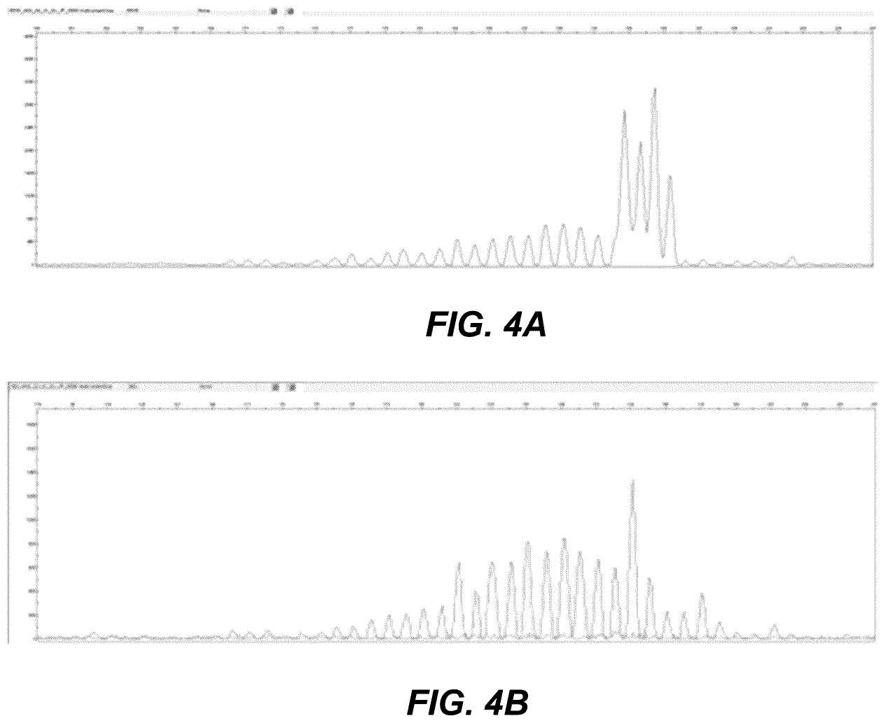

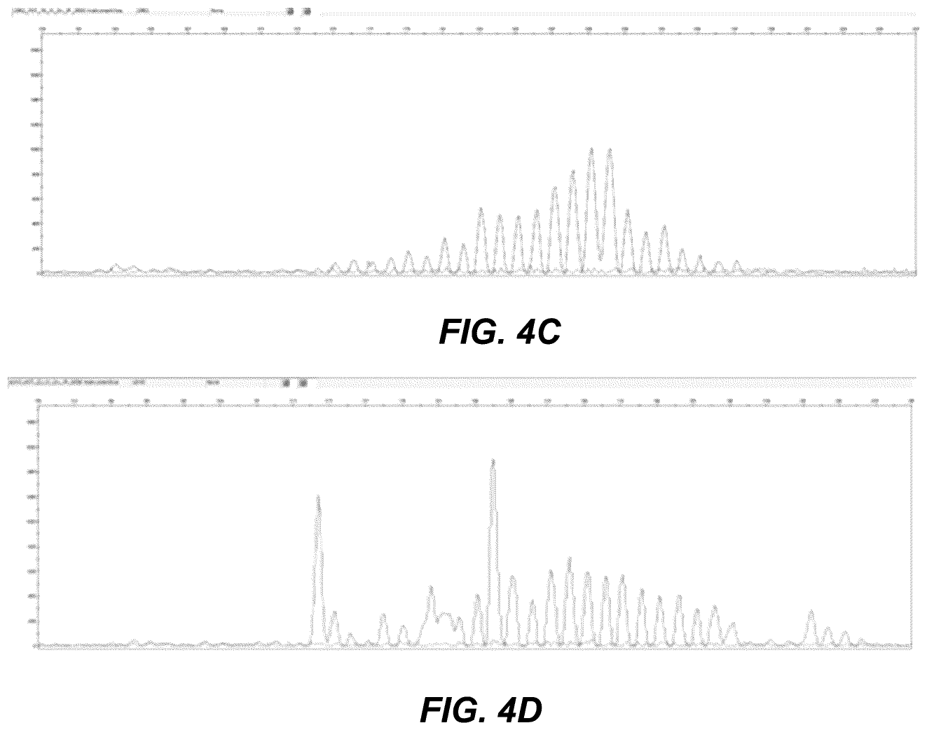

FIG. 4A through FIG. 4D illustrates various examples of graphical illustrations of datasets generated from an assay performed by the detection system 120, in accordance with some embodiments. In some embodiments, while the datasets being electropherogram datasets generated by a T-Cell Receptor Gamma Gene Rearrangement Assay as illustrated in FIG. 2 are used as the examples, other types of detection datasets may also be used in other embodiments.

In FIG. 4A and FIG. 4B, both samples have reference results positive. In FIG. 4C and FIG. 4D, both samples have reference results negative.

The electropherogram for each sample is the plot of peak size (in bp) and peak intensity (RFU of amplicons) within the peak size range 159-207 bp. In most samples, reference positive test results have at least one much higher peak (“Prominent Peak”) compared to the other peaks within the same sample, and in the reference negative test result, the overall peak profile resembles a bell shape distribution (Gaussian distribution) with no prominent peak sticking out of the pattern (See FIG. 4A and FIG. 4C). However, there were some samples where presence (or identification) or absence of a prominent peak was not obvious for reference positive result and reference negative result, respectively (See FIG. 4B and FIG. 4D). This observation confirms the need for developing an objective analysis method without need of visual confirmation. As discussed in further detail below in association with FIG. 5, an automated determination process is developed based on the peak height and peak size information according to the electropherogram dataset.

### Example Data Determination Process

FIG. 5 is a flowchart depicting an example process 500 of generating automated determination of homogeneity regarding a detection dataset, in accordance with some embodiments. Alternative embodiments may include more, fewer, or different steps from those illustrated in FIG. 5, and the steps may be performed in a different order from those illustrated in FIG. 5. While the data analytic device 140 is used as an example, these steps may be performed by any computing device. Additionally, each of these steps may be performed automatically by a computing device without human intervention. The process 500 may be embodied as a computer product that includes one or more computer-readable media that stores computer instructions that are executable by one or more processors. The instructions, when executed by the processors, cause the processors to perform various steps in the process 500.

In some embodiments, the data analytic device 140 receives 510 a dataset generated from detection analysis analyzing a sample. For example, the detection analysis may be an assay analyzing a biological sample of a subject, such as electropherogram and NGS. The assay may be a biological assay conducted using the detection system 120. The dataset may be generated by the signal generator 130. The biological sample may be any one of lymphoid tissue, a solid tumor biopsy, a tissue biopsy, FFPE, blood, whole blood, plasma, serum, urine, cerebrospinal fluid, fecal, saliva, tears, pleural fluid, pericardial fluid, or peritoneal fluid. Other suitable biological samples are also possible for analysis.

The process of conducting an assay may include obtaining the biological sample of the subject. The assay may be the assays described in FIG. 2 and FIG. 3 or another assay, such as an NGS. In some assays, one or more probes are added to a solution of the biological sample. The probes may take different forms and may include different types of probes. For example, in some situations, a probe may be a sequence labeled with a detectable marker, such as a fluorescent dye, used to bind to a specific DNA or RNA sequence to detect, quantify, or visualize the sequence. In some embodiments, a probe may also be a primer that a short sequence of nucleic acids that binds to a specific region of DNA or RNA to initiate replication or amplification, as in PCR. The primers may take the form of oligonucleotide primers with nucleotide sequences that are configured to hybridize with nucleotides of the biological sample in target genetic regions. In some embodiments, the primers are fluorescently labeled. The target genetic regions may include conserved genetic regions. For example, a target genetic region is a region within the T-cell receptor gamma chain gene. In some embodiments, the primers are included a single master mix. The mix contains primers that target the Vγ2, 3, 4, 5, 8, 9, 10 & 11, Jγ1/Jγ2, JγP, and JγP1/JγP2 regions.

While the T-cell receptor gamma chain gene is used as an explicit example in this disclosure, target genetic regions in other genes may also be analyzed by an assay and subsequent data analysis. Examples of genes that may be analyzed using the process 500 include but not limited to the following genes: ABL, ACVR1B, AKT3, AMER1, APC, ARID1A, ARID1B, ARID2, ASXL1, ASXL2, ATM, ATR, BAP1 BCL2, BCL6, BCORL1, BCR, BLM, BRAF, BRCA1, BTG1, CASP8, CBL, CCND3, CCNE1, CD74, CDC73, CDK12, CDKN2A, CHD2, CJD2, CREBBP, CSF1R, CTCF, CTNNB1, DICER1, DNAJB1, DNMT1, DNMT3A, DNMT3B, DOT1L, EED, EGFR, EIF1AX, EP300, EPHA3, EPHA5, EPHB1, ERBB2, ERBB4, ERCC2, ERCC3, ERCC4, ESR1, FAM46C, FANCA, FANCC, FANCD2, FANCE, FAT1, FBXW7, FGFR3, FLCN, FLT1, FLT3, FOXO1, FUBP1, FYN, GATA3, GPR124, GRIN2A, GRM3, H3F3A, HIST1H1C, IDH1, IDH2, IKZF1, IL7R, INPP4B, IRF4, IRS1, IRS2, JAK2, KAT6A, KDM6A, KEAP1, KIF5B, KIT, KLF4, KLH6, KMT2C, KRAS, LMAP1, LRP1B, LZTR1, MAP3K1, MCL1, MGA, MSH2, MSH6, MSTIR, MTOR, MYD88, NPM1, NRAS, NTRK1, NTRK2, NUP93, NUTM1, PAX3, PAX8, PBRM1, PGR, PHOX2B, PIK3CA, POLE, PTCH1, PTEN, PTPN11, PTPRT, RAD21, RAF1, RANBP2, RB1, REL, RFWD2, RHOA, RPTOR, RUNX1, RUNX1T1, SDHA, SHQ1, SLIT2, SMAD4, SMARCA4, SMARCD1, SNCAIP, SOCS1, SPEN, SPTA1, SUZ12, TET1, TET2, TGFBR, and/or TNFRSF14 and clonality detection in immunoglobulin gene rearrangements including immunoglobulin heavy chain (IGH) and light chain (IGK), or any T cell receptor gene rearrangement, such as T cell receptor beta (TRB), T cell receptor alpha (TRA), T cell receptor delta (TRD) and T cell receptor gamma (TRG).

The precise primers and sequence of the primers used in an assay depend on the target genetic regions and the target gene. A person with ordinary skill in the art would understand how to design the sequences of the primers. In one example, primers are designed to target conserved regions within the variable (V) and the joining (J) regions that flank the unique hypervariable antigen-binding region 3 (CDR3) or another complementarity determining region of a T cell receptor.

In some embodiments, the assay may include performing a polymerase amplification (e.g., polymerase chain reaction, PCR) to generate amplicons. For example, using the primers, the target genetic regions of the biological sample are amplified to generate amplicons. The amplicons can be of varying fragment sizes (e.g., base pair lengths or fragment sizes) due to gene rearrangement. For example, gene rearrangement is a regulated process in T-cell development enabling recognition of specific antigens. In some embodiments, the amplicons have an expected fragment size range. For example, the amplicons generated from analyzing the T-cell receptor gamma chain gene have an expected fragment size range of 159 bp to 207 bp.

In some embodiments, through the signal generator 130, a signal detection analysis is performed on the sample or amplified sample to generate the data for analysis by the data analytic device 140. For example, the detection analysis can be capillary electrophoresis or NGS. Amplifying the region with fluorescently labeled primers is followed by fractionation by capillary electrophoresis. The result of the signal detection analysis may take the form of a signal chart. In capillary electrophoresis, the detection-assay data may be an electropherogram dataset that includes a set of peaks corresponding to measurements of amplicons. While an electropherogram dataset is described, other embodiments may also use any suitable data representation of separated DNA fragments, such as a mass spectrogram, where each peak corresponds to a molecular fragment detected by mass spectrometry; a chromatogram from liquid chromatography, illustrating separated compounds; a fluorescence intensity plot, visualizing emission peaks from tagged molecules; an absorbance spectrum, displaying peaks of light absorption for identifying molecular structure; or an X-ray diffraction pattern, representing crystal lattice structures from X-ray diffraction analysis.

Each peak in the set corresponds to a fragment size and an intensity. While a fragment size is used as an example of a fragment size, other types of fragment sizes may also be analyzed in a similar manner. Peak intensity may be measured in the Relative Florescence Unit (RFU) in capillary electrophoresis. In some embodiment, the electropherogram dataset can be represented as a plot of peaks with the x-axis being the fragment size of the amplicons and the y-axis being the heights of the peaks measured by the intensity of each peak. Examples of plots of the electropherogram dataset are discussed and illustrated in FIG. 4A through FIG. 4D.

While electropherogram and capillary electrophoresis are used as explicit examples to illustrate the process 500, other datasets in any suitable format and data structure with other sets of data may also be used. Likewise, in analyzing the sample fragments (e.g., amplicons) after an amplification process, a method other than capillary electrophoresis may also be used to determine the fragment sizes and peak heights of the fragments corresponding to the measured fragment sizes. As such, while an electropherogram dataset is used as an example, another type of dataset that shows the distribution of the fragment sizes of the fragments may also be used in the process 500.

The detection dataset may include nucleic-acid measurements (fragment size data, actual determined nucleotide sequences) of the nucleic acid fragments. While the size data of amplicons is used as an example, in various embodiments, the nucleic-acid measurements do not need to be limited to size. Instead, the nucleic-acid measurements may encompass size data in electropherogram, actual determined nucleotide sequences in NGS, etc. Using size data as a example of the nucleic-acid measurement, a size data may take the form any population of nucleic acid fragments from a variety of sources, provided that the data reflects a distribution of fragment sizes relative to intensity. For example, the dataset may originate from fragmented genomic DNA following enzymatic digestion, where the analysis focuses on identifying predominant fragment sizes within the population. In some embodiments, the size data may also be data that are not related to nucleic acid fragments.

In some embodiments, the data analytic device 140 extracts 520 one or more features from the dataset. In the context of the electropherogram dataset, one or more features of the peaks in the electropherogram dataset may be extracted. Features can be any measurable attributes or metrics that are extracted from the data values, whether the features are raw or processed, absolute or normalized, qualitative or statistical, discrete or continuous, graphical or numerical, directly calculable or latent, individual or aggregated. In the context of NGS, the features may take the form of a version of a read count for a particular sequence read, such as a raw read count, a percentage read count, an aggregated read count, etc.

By way of example, a feature extracted from the dataset is an intensity value (e.g., peak height) of a peak corresponding to one or more target entities. Alternatively, or additionally, a feature extracted from the dataset is a systematically transformed value of a detection value (e.g. normalized detection value height/intensity). Alternatively, or additionally, a feature extracted from the dataset is an area under the curve (AUC) of a peak. Alternatively, or additionally, a feature extracted from the dataset is a number of detection values with values/intensities above a threshold value. Alternatively, or additionally, a feature extracted from the dataset is an aggregation of one or more detection values. Alternatively, or additionally, a feature extracted from the dataset is a statistical determination (e.g., average, standard deviation, variance) of detection value intensities of one or more detection values. Alternatively, or additionally, a feature extracted from the dataset is an intensity ratio of a detection value relative to a reference (e.g., an average value of the rest of the detection values). Alternatively, or additionally, a feature extracted from the dataset is a modeling metric of one or more detection values relative to a distribution (e.g., metrics in Kolmogorov-Smirnov test). For example, the feature may measure how well the detection values are fit to a known distribution such as whether the detection value distribution is well described by a Gaussian distribution. Alternatively, or additionally, a feature extracted from the dataset is a pattern recognition of the overall profile of the peaks.

In some embodiments, the extraction of one or more features includes determining a normalized peak intensity. By way of example, the data analytic device 140 selects a representative peak from the peaks in the electropherogram dataset. The selection is carried out according to one or more selection criteria. For example, the selection criteria may be selecting the highest peak, the lowest peak, the top N highest peaks (e.g., N=1, 2, 3, 4, 5, 6, 7, 8, 9, 10, 20, 30, 40, 50), the top N lowest peaks, an average peak, a peak at certain a percentile, peaks above or below a threshold, etc. The data analytic device 140 in turn determines the intensity of a selected representative peak. The data analytic device 140 also determines a peak normalization reference based on aggregating one or more peaks in the set. In some embodiments, the peak normalization reference may be an average value of the peaks in the set. In some embodiments, the peak normalization reference may be an average value of the peaks in the set but excluding one or more highest peaks. For example, in some embodiments, the peak normalization reference may be the average value of the peaks excluding the two highest peaks. The data analytic device 140 determines a normalized peak intensity of the representative peak by dividing the intensity of the selected representative peak by the peak normalization reference. In some embodiments, one or more extracted features of the peaks include the normalized peak intensity. In some embodiments, one or more extracted features include an aggregation or statistics of the normalized peak intensities of more than one selected representative peak. The peak normalization reference may also be referred to as a detection-value-normalization reference.

By way of example, in some embodiments, the maximum normalized height is used as an extracted feature. Since the absolute value of the peak intensity (measured RFU) tends to vary from instrument to instrument and run to run, peak height normalization is adopted. When normalizing, in order to accentuate the difference between the highest peak and other peaks, the peak height normalization is calculated by individual peak height divided by the sum of applicable peak heights. In some embodiments, the first two highest peaks are excluded when calculating the peak normalization reference. This is because some clonal samples are bi-allelic, with the two highest peaks with similar RFU. The exclusion of the two highest peaks from the denominator of the normalizing equation causes the highest peak to have a greater normalized value in clonal positive samples. The exclusion exhibits negligible effect on the clonal negative samples since peak heights of the first two highest peaks in the clonal negative samples will be similar to the rest of the peaks. Hence, this normalized peak height from positive samples is more easily differentiated from negative samples. The highest normalized peak height is referred to as the maximum normalized height or MNH.

In some embodiments, the extraction of one or more features includes classifying a peak to a band based on the fragment size of the peak. By way of example, the data analytic device 140 selects a representative peak from the peaks in the electropherogram dataset according to one or more selection criteria. The selection criteria may be selecting the highest peak, the lowest peak, the top N highest peaks, the top N lowest peaks, an average peak, a peak at certain a percentile, peaks above or below a threshold, etc. The data analytic device 140 determines the fragment size of a representative peak. For example, the representative peak can be the peak with the highest intensity or the maximum normalized height. The data analytic device 140 classifies the representative peak into a band according to the fragment size of the representative peak. In some embodiments data analytic device 140 may maintain a plurality of bands. Each band has a corresponding fragment size range. A peak that falls within the fragment size range is assigned to the corresponding band. In some embodiments, the total range of the various bands is based on the expected fragment size range of the amplicons. This expected fragment size range can be divided into multiple bands.

In some embodiments, the expected fragment size range depends on the primers used in the assay, the target genetic regions, and the target gene. In some embodiments, the range varies because of gene rearrangement for the target genetic regions but may be estimated based on empirical data. In some embodiments, the amplicons resulting from analyzing the T-cell receptor gamma chain gene have an expected fragment size range of 159 bp to 207 bp. In some embodiments, this expected fragment size range is divided into three or more bands, and features are determined based on the band within which the peak's fragment size falls.

In some embodiments, the extraction of one or more features includes generating a feature vector that can be used as an input to a machine learning model. For example, the feature vector includes a plurality of values of features that are described in this disclosure. The feature values may be arranged in a structured manner and one or more normalization techniques may be used to adjust the features within the vector. The feature vector includes multiple dimensions, and each dimension corresponds to one of the features.

In some embodiments, in the context of NGS, the extraction of one or more features includes determining a normalized read counts for certain reads but excluding the highest read counts. By way of example, the data analytic device 140 identifies sequence reads that are from target genes, such as the V-gene and J-gene. For example, after sequencing, the data analytic device 140 may map the sequence reads to a reference genome to identify a subset of sequence reads that are determined to be part of the V-gene and/or J-gene. Those sequence reads in the subset can be sorted based on the read counts. For each sequence read, a percentage read count may be determined by having the raw read count corresponding to the sequence read divided by the total read counts of sequence reads. Top N (e.g., N=1, 2, 3, 4, 5, 6, 7, 8, 9, 10, 20, 30, 40, 50) sequence reads that have the highest percentage read counts can be excluded. Excluding the top N sequence reads, the remaining sequence reads with the highest read count may be normalized. For example, for the remaining sequence reads, the total read counts can be determined by summing all of the read counts correspond to the remaining sequence reads. This can be used as a detection-value-normalization reference.

In some embodiments, the data analytic device 140 inputs 530 the one or more features of the peaks into a model, such as a model 142. In some embodiments, the one or more features are a single feature. In some embodiments, the single feature is the maximum normalized height. In some embodiments, a plurality of features may be used. For example, a feature vector that includes multiple feature values is used.

In various embodiments, the model may take one of various suitable forms. For example, in some embodiments, the model is a rule-based model that includes one or more classification rules used to analyze one or more features. The rules can be arranged in parallel, hierarchically in a decision tree, and/or independently to generate a determination. The determination may be based on scoring various classification rules, going through a decision tree, comparing one or more values generated by the rules to one or more thresholds, or another suitable method. In some embodiments, the model may be a heuristic model that makes a determination based on a combination of rules, heuristics, constraints, factors, and algorithms. In some embodiments, the model is a machine learning model that is trained using one or more artificial intelligence training techniques. In some embodiments, the model is a statistical model that applies one or more statistical techniques (e.g., determining probabilities, likelihoods, p-values, etc.) and uses one or more statistical distributions. In some embodiments, the model is a regression model that applies one or more regression techniques. In some embodiments, the model is a composite model that includes more than one instance of a type of model and/or that includes a combination of various types of models.

In some embodiments, the model is trained based on training samples of past datasets of homogeneous and non-homogeneous samples, such as past electropherogram datasets of clonal samples and non-clonal samples. For example, some biological samples are known to be clonal and the electropherogram datasets generated by analyzing those biological samples may be used as positive training samples. Electropherogram datasets corresponding to other biological samples that are known to be non-clonal may be used as negative training samples. Training may be performed in various ways in different embodiments. In some embodiments, machine learning techniques, such as forward propagation, backpropagation, and coordinate descent, may be applied in training. In some embodiments, training may be referred to as determining coefficients of one or more regression models using the training samples' data points. In some embodiments, training may be referred to as determining one or more classification rules based on analyzing the training samples. Other ways to perform training are also possible.

By way of example, in some embodiments, training of the model includes collecting the training samples. In the context of a biological assay, the training samples may be generated from assays that apply one or more primers that target conserved genetic regions to biological samples. In turn, electropherogram datasets are generated from the results of the assays. The training samples are labeled based on the clonality of the biological samples. The data analytic device 140 may adjust one or more parameters in the model based on the training samples.

In some embodiments, a model is trained specifically for an assay based on one or more factors of the assay. For example, the factors of an assay may include the nature of the assay, the primers, molecules, and other reagents used in the assay, the biological sample, the target genetic regions, and the target gene. The training samples may be generated using the same primers that are used in analyzing the biological sample of a subject. As such, the expected fragment size range of the amplicons is the same for the training samples and the subject's sample. A model specific to the factors of the assay is trained so that the model can accurately predict and analyze the result of the subject's sample. For example, the electropherogram dataset corresponding to the biological sample of the subject is generated from an assay that applies the same one or more primers used in generating the training samples. Training for NGS dataset may be similar using conditions specific to the NGS.

In some embodiments, training techniques in machine learning may be used in conducting training of the model. By way of example, the data analytic device 140 initiates one or more parameters of the model. The data analytic device 140 receives the training samples of the past electropherogram datasets of clonal samples and non-clonal samples. The training samples are associated with clonality labels. The data analytic device 140 applies, in forward propagation, the model to predict the clonality of one or more training samples. In turn, the data analytic device 140 compares the predicted clonality to the clonality labels of the training samples. The data analytic device 140 adjusts, in backpropagation, the one or more parameters based on the comparison.

In some embodiments, the input of the model may be size range band-specific. For example, the model may include a plurality of sub-models. Each sub-model corresponds to a band. In response to a feature being assigned to a band, the data analytic device 140 selects a sub-model corresponding to the selected model and inputs the extracted feature to the sub-model. By way of the example, the data analytic device 140 identifies a peak from which one of the features is extracted or a read count from which one of the features is extracted. The data analytic device 140 determines the fragment size of the identified peak or the nucleic acid sequence corresponding to the identified read count. The data analytic device 140 classifies the identified peak into a band according to the fragment size of the identified peak or according to the nucleic acid sequence. In turn, the data analytic device 140 applies a band-specific regression model selected from a set of regression models. The regression models in the set correspond to the set of bands. The applied band-specific regression model corresponds to the band to which the identified peak is classified or the band to which the identified read count is classified. The data analytic device 140 generates a score based on the band-specific regression model.

In some embodiments, at least two of the regression models in the set include offset values that are offset from each other. For example, it was found that the positive control for the assay analyzing the T-cell receptor gamma chain gene generates the highest peak in the range of 192.99 bp to 193.12 bp. Empirical results show that there are instances when the fragment size is 192.99 bp resulting in non-clonal results, causing the positive control to be invalid. The classification results from both the PB training dataset and test data (PB Test dataset and FFPE Test dataset for validation) show no difference if a cutoff is adjusted from 183 and 193 to 183 and 192.5 in the model.

In some embodiments, MNH and band values from a training dataset that includes training samples (“PB Trainer dataset”) are calculated and used to train the prediction model. In some embodiments, the model is a composite that includes a set of band-specific regression models. The model may be referred to as a regression-based predictor (RBP). The RBP is the MNH value (based on peak intensity) transformed in one of three ways, based on the accompanying band value (based on peak size and cutoff 183 and 192.5). In one example, three band-specific MNH transforming models are described and used. In other embodiments, other numbers of band-specific models may be used.
RBP=Intercept+beta*MNH if band=1;
RBP=Intercept+beta*MNH+offset_band_2 if band=2;
RBP=Intercept+beta*MNH+offset_band 3 if band=3;
Cutoff=threshold, if RBP>cutoff then binary predictor=1, classified as positive otherwise binary predictor=0, classified as negative.

In some embodiments, precise values of the intercept coefficient, beta coefficient, band-specific offsets, and threshold can be determined during training. The values may depend on the training samples. In some embodiments, the values may also depend on factors of the assay, such as the nature of the assay, the primers, molecules, and other reagents used in the assay, the biological sample, the target genetic regions, and the target gene. In some embodiments, a specific model is trained for each particular type of assay.

The beta value may any numerical value, including negative value.

In some embodiments, the data analytic device 140 generates 540 a computer-automated determination of homogeneity of the sample based on an output of the model. In some embodiments, homogeneity may refer to clonality of the sample, but in other embodiments homogeneity may encompass other concepts. The homogeneity may indicate a concentrated population of nucleic acid fragments that share a similar size, signaling a predominant peak within the data. While homogeneity may arise from clonal populations, where identical copies of a sequence are amplified, homogeneity can also result from non-clonal sources, such as in situations where fragments are uniform in size but not necessarily identical in sequence. In some embodiments, the data analytic device 140 identifies homogeneity based on size distribution without requiring clonal origin, allowing a detection of any predominant population of size-consistent nucleic acid molecules, regardless of their underlying sequence characteristics.

By way of example, the data analytic device 140 receives a score from the model. The data analytic device 140 compares the score to a threshold to determine whether the biological sample of the subject is clonal or non-clonal. Scores landing on one side of the threshold are defined to be clonal while scores on the other side of the threshold are defined to be non-clonal. In some embodiments, alternatively to or in addition to a binary determination of whether the biological sample is clonal or non-clonal, a model may be trained to identify a particular clone in the sample. In some embodiments, clonality for certain genetic regions has a high correspondence of malignancy (e.g., >85%). In some embodiments, the process 500 may be used to determine or predict malignancy.

While process 500 is described as an example of a process that can be used to determine the clonality of a biological sample, the process 500 may also be used outside of the biological assay setting and as a process to determine whether a dataset follows a particular statistical distribution such as the Gaussian distribution. Likewise, the distribution may not be Gaussian, such as in the situation of certain next-generation sequencing data.

### Example Process for Cancer Diagnosis

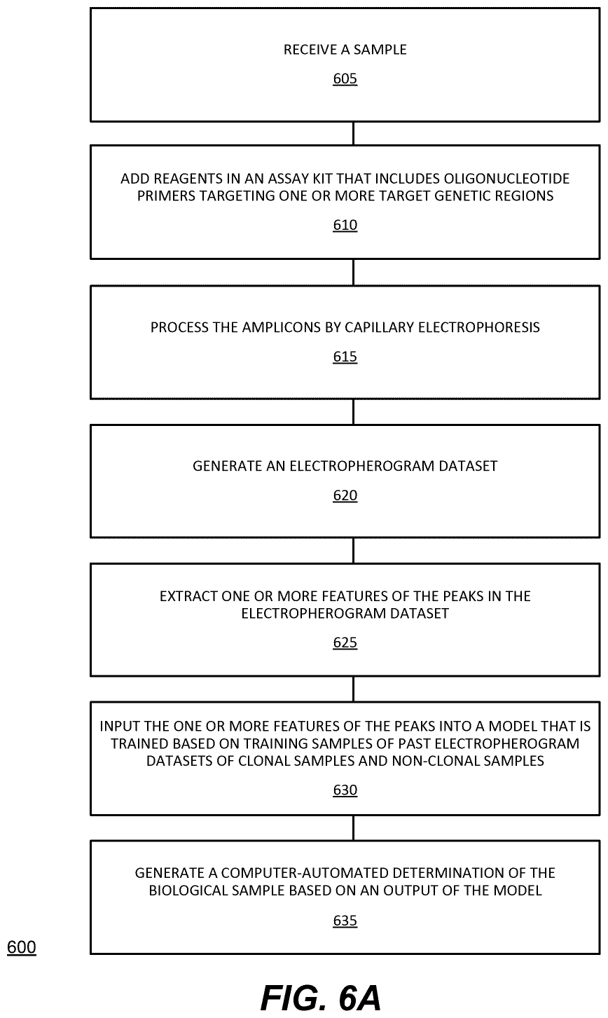

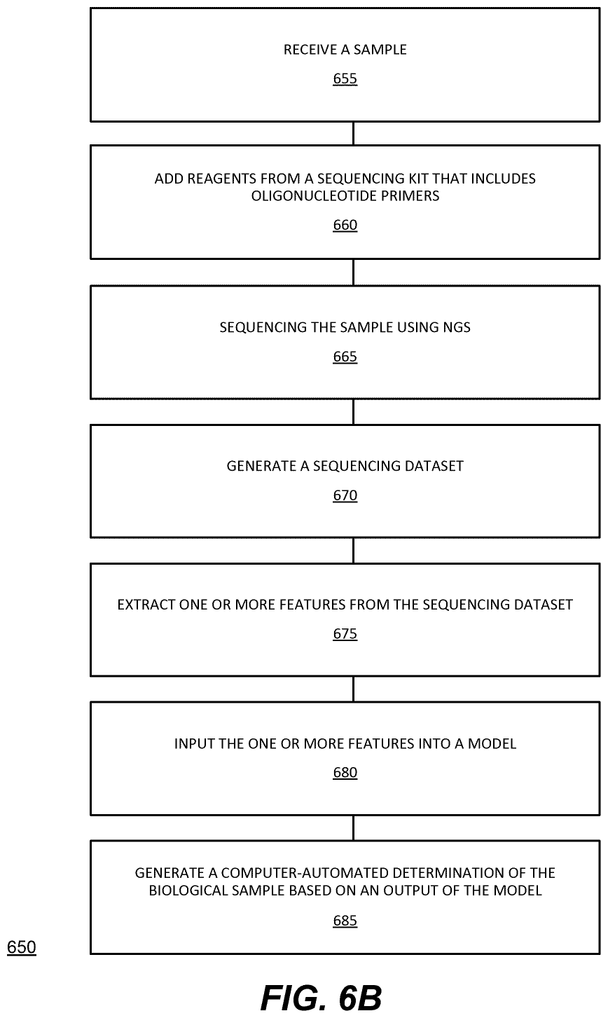

FIG. 6A is a flowchart depicting an example process 600 of generating a computer-automated determination regarding the malignancy of a biology sample in cancer diagnosis, in accordance with some embodiments. Alternative embodiments may include more, fewer, or different steps from those illustrated in FIG. 6A, and the steps may be performed in a different order from those illustrated in FIG. 6A. While some of the steps are described as using the data analytic device 140, those computer-implemented steps may be performed by any computing device. Some of the computer-implemented steps in process 600 may be embodied as a computer product that includes one or more computer-readable media that stores computer instructions that are executable by one or more processors. The instructions, when executed by the processors, cause the processors to perform those computer-implemented steps. Various details and examples that are discussed in this disclosure, such as in FIG. 5, are incorporated herein for the discussion of the process 600.

The process 600 includes receiving 605 a biological sample. The biological sample may be processed by one or more conventional steps before a solution of the biological sample is generated and ready for polymerase chain reaction (PCR). For example, those preparation steps may include homogenization that disrupts the tissue sample to break down cells and release DNA, DNA extraction using phenol-chloroform extraction or other commercial DNA extraction kits, purification, quantification that measures the contraction of DNA in the purified sample, and dilution. The biological sample may be a body fluid sample such as blood or tissue sample, or any suitable biological sample.

The process 600 includes adding 610 reagents in an assay kit that includes oligonucleotide primers targeting one or more target genetic regions. In some embodiments, the oligonucleotide primers are fluorescently labeled. The oligonucleotide primers are configured to hybridize with nucleotides of a biological sample in the target genetic regions to generate, through an amplification process such as PCR, a set of amplicons with an expected fragment size range. In some embodiments, the primers are designed to target conserved regions within the variable (V) and the joining (J) regions that flank the unique hypervariable antigen-binding region 3 (CDR3).

The process 600 includes processing 615 the amplicons by capillary electrophoresis. The amplicon sample solution may be injected into a capillary tube and injected into a capillary electrophoresis machine. The capillary electrophoresis machine applies electric current to separate the amplicons according to the fragment sizes. An excitation source is used to excite the fluorescent labels of the amplicons. A detector is used to record the intensity of the fluorescent signals.

The process 600 includes generating 620 a detection dataset, such as an electropherogram dataset. While an electropherogram dataset is used as an example here, the process 600 may also be used for other type of signal charts. The electropherogram dataset may take the form of a plot that shows the intensity of the fluorescent signal (y-axis) as a function of fragment size (x-axis). Each peak in the electropherogram corresponds to a different amplicon size. The position of each peak on the x-axis is related to the fragment size, and the peak height or area is proportional to the amount of that amplicon size in the sample.

The process 600 includes extracting 625 one or more features of the peaks in the electropherogram dataset. In some embodiments, at least one feature is determined from the intensity of a particular peak. One of the features may be a normalized maximum height in the electropherogram dataset.

The process 600 includes inputting 630 the one or more features of the peaks into a model that is trained based on training samples of past electropherogram datasets of clonal samples and non-clonal samples. The model may be a machine learning model, heuristic model, rule-based model, a regression model and/or a combination thereof.

The process 600 includes generating 635 a computer-automated determination of the biological sample based on an output of the model. The determination may be the clonality of the biological sample. The determination may also be the malignancy of the sample in cancer diagnosis. For example, the process 600 may be used as a confirmation test.

FIG. 6B is a flowchart depicting an example process 650 of generating a computer-automated determination regarding the malignancy of a biology sample in cancer diagnosis, in accordance with some embodiments. Alternative embodiments may include more, fewer, or different steps from those illustrated in FIG. 6B, and the steps may be performed in a different order from those illustrated in FIG. 6B. While some of the steps are described as using the data analytic device 140, those computer-implemented steps may be performed by any computing device. Some of the computer-implemented steps in process 650 may be embodied as a computer product that includes one or more computer-readable media that stores computer instructions that are executable by one or more processors. The instructions, when executed by the processors, cause the processors to perform those computer-implemented steps. Various details and examples that are discussed in this disclosure, such as in FIG. 5, are incorporated herein for the discussion of the process 650.

The process 650 is similar to the process 600, except NGS is used as the detection assay and the detection dataset being analyzed is sequencing dataset that includes sequence reads and corresponding base counts. Things that are similar to the process 600 are not repeatedly discussed.

The process 650 includes receiving 655 a biological sample. The process 650 includes adding 660 reagents from a sequencing kit that includes oligonucleotide primers. The process 650 includes sequencing the sample using NGS. The process 650 includes generating 670 a detection dataset, such as a sequencing dataset. The process 650 includes extracting 675 one or more features in the sequencing dataset. Detail of examples of features extraction is discussed in FIG. 5. For example, the data analytic device 140 may map the sequence reads to a reference genome and extract a subset of sequence reads that are mapped to gene of interest, such as the V-gene or the J-gene. The data analytic device 140 may then in turn extract features from the subset of the sequence reads. In some embodiments, at least one feature is determined from a normalized read count of a particular sequence read. The process 650 includes inputting 680 the one or more features into a model. The process 650 includes generating 685 a computer-automated determination of the biological sample based on an output of the model. The determination may be the clonality of the biological sample. The determination may also be the malignancy of the sample in cancer diagnosis. For example, the process 650 may be used as a confirmation test.

### Example Model Training and Structure

In various embodiments, a wide variety of machine learning techniques may be used. Examples include different forms of supervised learning, unsupervised learning, and semi-supervised learning such as decision trees, support vector machines (SVMs), regression, Bayesian networks, and genetic algorithms. Deep learning techniques such as neural networks, including convolutional neural networks (CNN), recurrent neural networks (RNN), and long short-term memory networks (LSTM), may also be used. For example, various prediction tasks of whether an assay dataset indicates clonality of a biological sample performed by a model 142 as discussed in process 500 and process 600, determination task of whether a dataset follows a statistical distribution such as the Gaussian distribution, and other processes may apply one or more machine learning and deep learning techniques.

In various embodiments, the training techniques for a machine learning model may be supervised, semi-supervised, or unsupervised. In supervised learning, the machine learning models may be trained with a set of training samples that are labeled. For example, for a machine learning model trained to make a prediction of the clonality of a biological sample, the training samples may be past datasets generated from biological samples of known clonality. The labels for each training sample may be binary or multi-class. In training a machine learning model for clonality prediction, the training labels may include a positive label indicating that the training sample dataset was generated from a biological sample that is clonal and a negative label indicating that the training sample dataset was generated from a biological sample that is non-clonal. In some embodiments, the training labels may also be multi-class or a continuous score. For example, the training labels may correspond to a particular clone. In another example, the training labels may be scores that indicate a degree of clonality or malignancy.

By way of example, the training set may include multiple known records of datasets generated by biological samples with known natures such as known clonality or malignancy. Each training sample in the training set may correspond to a past and the corresponding outcome may serve as the label for the sample. A training sample may be represented as a feature vector that includes multiple dimensions. Each dimension may include data of a feature, which may be a quantized value of an attribute that describes the past record. For example, in a machine learning model that is used to predict the clonality of a biological sample, the features in a feature vector may include the maximum normalized intensity of the dataset, the area under the curve, other features that are described in the process 500, etc. In various embodiments, certain pre-processing techniques may be used to normalize the values in different dimensions of the feature vector.

In some embodiments, an unsupervised learning technique may be used. The training samples used for an unsupervised model may also be represented by features vectors, but may not be labeled. Various unsupervised learning techniques such as clustering may be used in determining similarities among the feature vectors, thereby categorizing the training samples into different clusters. In some cases, the training may be semi-supervised with a training set having a mix of labeled samples and unlabeled samples.

A machine learning model may be associated with an objective function, which generates a metric value that describes the objective goal of the training process. The training process may intend to reduce the error rate of the model in generating predictions. In such a case, the objective function may monitor the error rate of the machine learning model. In a model that generates predictions, the objective function of the machine learning algorithm may be the training error rate when the predictions are compared to the actual labels. Such an objective function may be called a loss function. Other forms of objective functions may also be used, particularly for unsupervised learning models whose error rates are not easily determined due to the lack of labels. In some embodiments, in a model that is used to predict clonality, the objective function may correspond to a loss function that compares the predicted clonality using the model and the actual clonality labels. In various embodiments, the error rate may be measured as cross-entropy loss, L1 loss (e.g., the sum of absolute differences between the predicted values and the actual value), L2 loss (e.g., the sum of squared distances).

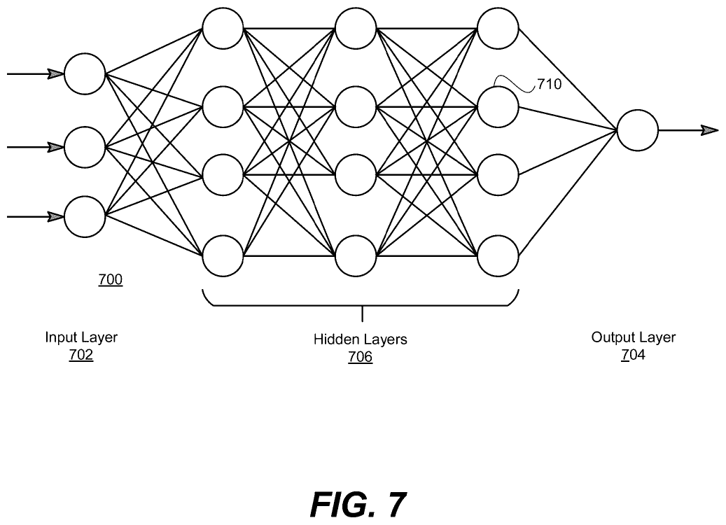

Referring to FIG. 7, the structure of an example neural network is illustrated, in accordance with some embodiments. The neural network 700 may receive an input and generate an output. The input may be the feature vector of a training sample in the training process and the feature vector of an actual case when the neural network is making an inference. The output may be the prediction, classification, or another determination performed by the neural network. The neural network 700 may include different kinds of layers, such as convolutional layers, pooling layers, recurrent layers, fully connected layers, and custom layers. A convolutional layer convolves the input of the layer (e.g., an image) with one or more kernels to generate different types of images that are filtered by the kernels to generate feature maps. Each convolution result may be associated with an activation function. A convolutional layer may be followed by a pooling layer that selects the maximum value (max pooling) or average value (average pooling) from the portion of the input covered by the kernel size. The pooling layer reduces the spatial size of the extracted features. In some embodiments, a pair of convolutional layers and a pooling layer may be followed by a recurrent layer that includes one or more feedback loops. The feedback may be used to account for spatial relationships of the features in an image or temporal relationships of the objects in the image. The layers may be followed by multiple fully connected layers that have nodes connected to each other. The fully connected layers may be used for classification and object detection. In some embodiments, one or more custom layers may also be presented for the generation of a specific format of the output. For example, a custom layer may be used for image segmentation for labeling pixels of an image input with different segment labels.

The order of layers and the number of layers of the neural network 700 may vary in different embodiments. In various embodiments, a neural network 700 includes one or more layers 702, 704, and 706. A machine learning model may include certain layers, nodes 710, kernels and/or coefficients. Training of a neural network, such as the NN 700, may include forward propagation and backpropagation. Each layer in a neural network may include one or more nodes, which may be fully or partially connected to other nodes in adjacent layers. In forward propagation, the neural network performs the computation in the forward direction based on the outputs of a preceding layer. The operation of a node may be defined by one or more functions. The functions that define the operation of a node may include various computation operations such as convolution of data with one or more kernels, pooling, recurrent loop in RNN, various gates in LSTM, etc. The functions may also include an activation function that adjusts the weight of the output of the node. Nodes in different layers may be associated with different functions.

Training of a machine learning model may include an iterative process that includes iterations of making determinations, monitoring the performance of the machine learning model using the objective function, and backpropagation to adjust the weights (e.g., weights, kernel values, coefficients) in various nodes 710. For example, a computing device may receive a training set that includes past electropherogram datasets of clonal samples and non-clonal samples. Each training sample in the training set may be assigned with labels indicating clonality. The computing device, in forward propagation, may use the machine learning model to generate the predicted clonality of one or more training samples. The computing device may compare the predicted clonality with the labels of the training sample. The computing device may adjust, in a backpropagation, the weights of the machine learning model based on the comparison. The computing device backpropagates one or more error terms obtained from one or more loss functions to update a set of parameters of the machine learning model. The backpropagation may be performed through the machine learning model and one or more of the error terms based on a difference between a label in the training sample and the generated predicted value by the machine learning model.

By way of example, each of the functions in the neural network may be associated with different coefficients (e.g., weights and kernel coefficients) that are adjustable during training. In addition, some of the nodes in a neural network may also be associated with an activation function that decides the weight of the output of the node in forward propagation. Common activation functions may include step functions, linear functions, sigmoid functions, hyperbolic tangent functions (tanh), and rectified linear unit functions (ReLU). After an input is provided into the neural network and passes through a neural network in the forward direction, the results may be compared to the training labels or other values in the training set to determine the neural network's performance. The process of prediction may be repeated for other samples in the training sets to compute the value of the objective function in a particular training round. In turn, the neural network performs backpropagation by using gradient descent such as stochastic gradient descent (SGD) to adjust the coefficients in various functions to improve the value of the objective function.

Multiple rounds of forward propagation and backpropagation may be performed. Training may be completed when the objective function has become sufficiently stable (e.g., the machine learning model has converged) or after a predetermined number of rounds for a particular set of training samples. The trained machine learning model can be used for performing clonality prediction tasks or another suitable task for which the model is trained.

In various embodiments, the training samples described above may be refined and continue to re-train the model, which is the model's ability to perform the inference tasks. In some embodiments, this training and re-training process may repeat, which results in a computer system that continues to improve its functionality through the use-retraining cycle. For example, after the model is trained, multiple rounds of re-training may be performed. The process may include periodically retraining the machine learning model. The periodic retraining may include obtaining an additional set of training data, such as through other sources, by usage of users, and by using the trained machine learning model to generate additional samples. The additional set of training data and later retraining may be based on updated data describing updated parameters in training samples. The process may also include applying the additional set of training data to the machine learning model and adjusting the parameters of the machine learning model based on the application of the additional set of training data to the machine learning model. The additional set of training data may include any features and/or characteristics that are mentioned above.

### EXAMPLES

In some embodiments, the TRG assay described in FIG. 2 employs multiple consensus DNA primers that target conserved genetic regions within the T-cell receptor gamma chain gene. It is designed to detect at least a majority of potential T-cell receptor gamma chain gene rearrangements. DNA from sample and/or run controls are amplified (e.g., PCR) to generate amplified products (amplicons) with expected sizes ranging from 159 to 207 bp. Amplicons are then processed on a signal generator 130 for capillary electrophoresis-mediated fragment analysis. The capillary electrophoresis results are stored in a suitable file format, such as a .fsa file format, which can be viewed as an electropherogram (electropherogram). electropherogram is a visual representation of the amplicons from that sample where the amplicons are separated by size and displayed as “peaks”, with peak heights representing the intensity of the associated amplicon. Peak size is measured in bp; peak intensity is measured in Relative Florescence Unit (RFU). For this assay, peak height RFU value is utilized, not peak area. The same data can also be viewed in .csv file format.

An algorithm may be used to predict the clonal and non-clonal status based on electropherogram corresponding data from samples. Since peak height was used in the model and the absolute value of the peak height (measured RFU) tends to vary between instrument to instrument and run to run, peak height normalization was adopted. When normalizing, in order to accentuate the difference between the highest peak and other peaks, the peak height normalization was calculated by individual peak height divided by the sum of all applicable peak heights except the first two highest peaks. This is because some TRG clonal samples are biallelic, with the two highest peaks with similar RFU. Exclusion of the two highest peaks from the denominator of the normalizing equation will cause the highest peak to have a greater normalized value in clonal positive samples. The exclusion will exhibit a negligible effect on the clonal negative samples since the peak heights of the first two highest peaks in the clonal negative samples will be similar to the rest of the peaks. So this normalized peak height from positive samples will be more easily differentiated from negative samples. The highest normalized peak height is referred to as the Maximum Normalized Height or MNH.

Since the main information is normalized peak height and corresponding peak size, the classification method based on the above information is desired. Various classification methods may be employed in various embodiments. In some embodiments, a logistic linear regression model is in model 142. A logistic linear regression model is used to classify the clonality status into clonality or non-clonality by using the ROC method. ROC is the plot of true positive rate (sensitivity) vs 1−specificity at different cutoffs for binary classification. By the ROC curve, usually, the best cutoff can be selected to have the best prediction result based on the study purpose. In some embodiments, one or more models may be built.

While the ROC model is discussed as an example, any suitable model can be used in various embodiments. In some embodiments, a Latent Variable Analysis (LVA) model can be employed to identify underlying patterns and reduce dimensionality in complex datasets. In some embodiments, a Spectral Decomposition Analysis (SDA) model can analyze signal intensities and distributions by breaking down data into fundamental components. In addition to LVA and SDA, other models may also be suitable depending on the analysis requirements. For example, Principal Component Analysis (PCA) can be used to simplify data and identify major variance components in high-dimensional datasets, which aids in detecting homogeneous populations.

In some embodiment, a first example model (Model I) is fit the logistic linear regression model using Diagnosis (Reference) vs. MNH only. Based on the training dataset, an AUC value was generated and a cutoff value was selected.

In some embodiments, the second example model (Model II) includes both MNH and the corresponding peak size (in bp) to fit the logistic linear regression model using the same dataset. The model fit improved, with an increased AUC. A cutoff value was selected based on the model training. However, PPA decreased.

In some embodiments, the third example model (Model III) is fitted using MNH and MNH peak size bands, where peak size are categorized into 3 different “bands” (categories) based on two different peak size cutoffs. The cutoffs are chosen based on the grid search by 1 bp change using the training data (PB training dataset) to get the best classification results based on ROC analysis. A cutoff value for peak size was selected to use. The AUC value increased. In some embodiments with other types of datasets or other assays, another model may be selected.

MNH and band values from more than 150 training sample data (“PB Trainer dataset”) were calculated and used to find the prediction model, which resulted in creating the new analysis method, with the final result determining value named RBP (Regression Based Predictor). RBP is MNH value (based on peak height) transformed in one of three ways, based on the accompanying band value (based on peak size and appropriate cutoff.5). Three band-specific MNH transforming equations are described below.

$$RBP = value\_A + value\_B * MNH if band = 1 ; \\ RBP = value\_A + value\_B * MNH + value\_C if band = 2 ; \\ RBP = value\_A + value\_B * MNH + value\_D if band = 3 ;$$

Cutoff version 01, if RBP>cutoff then binary predictor=1, classified as positive o/w binary predictor=0, classified as negative.

For simplicity, the model was reformatted to make cutoff=0 as follows:

$$RBP = adjusted\_value \_A + value\_B * MNH if band = 1 ; \\ RBP = adjusted\_offset \_band2 + value\_B * MNH if band = 2 ; \\ RBP = adjusted\_offset \_band3 + value\_B * MNH if band = 3 ;$$

Cutoff version 02, if RBP>Cutoff then binary predictor=1, classified as positive (Clonal);
otherwise, binary predictor=0, classified as negative (Non-clonal).

The model above was validated on two or more different types of datasets, such as a PB Test dataset and a FFPE dataset. Both validations show effective results. Classification results are shown in Table 1. With PPA-82.6% and 95% CI is (61.2%, 95.0%); NPA=94.4% and 95% CI is (84.6%, 98.8%); OPA=90.9% and 95% CI is (82.2%, 96.3%).

TABLE 1
Classification Results from PB Lymphoma Test Data

Reference Method
(Diagnosis)

| | | Positive | Negative | Total |
|---|---|---|---|---|
| IC TRG | Positive | 19 | 3 | 22 |
| | Negative | 4 | 51 | 55 |
| | Total | 23 | 54 | 77 |

The model is applied to the FFPE dataset (“FFPE Test dataset”) and classification results are shown in Table 2. With PPA-87.8% and 95% CI is (75.2%, 95.4%); NPA=90.2% and 95% CI is (78.6%, 96.7%); OPA=89.0% and 95% CI is (81.1%, 94.3%). A lower limit of 95% CI greater than 70% was observed for both PPA and NPA for this dataset.

TABLE 2
Classification Results from FFPE Data

Reference Method
(Diagnosis)

| | | Positive | Negative | Total |
|---|---|---|---|---|
| IC TRG | Positive | 43 | 5 | 48 |
| | Negative | 6 | 46 | 52 |
| | Total | 49 | 51 | 100 |

In one embodiment, an analysis was conducted on IC immunoglobulin heavy chain (IGH) electropherogram data. The IC IGH assay results were obtained by utilizing three separate master mixes, designated MMA, MMB, and MMC, where results were generated collectively from these three components. The results for each master mix—MMA, MMB, and MMC—were individually derived from data specific to each respective mix. Accordingly, in this embodiment, an algorithmic model method similar to the initial approach was applied separately to the data from each of the three master mixes.

Since the current method yields results for each of the three master mixes independently for each sample, the outputs of this method served as a reference for comparison. In this embodiment, the algorithmic model was first applied to the MMA training data to generate the model, which was then applied to both MMB and MMC training data, as well as to test data from all three master mixes.

In one embodiment, the concordance table results from the comparison of test method results with current method results are presented in Table 3. Table 3 included the concordance test results from training data for MMB and MMC, as well as test data for MMA, MMB, and MMC. Table 4 included the corresponding positive percent agreement (PPA), negative percent agreement (NPA), and 95% confidence intervals.

In one embodiment, the term “Neg” in the table indicated results identified as “Negative,” while “Pos” indicated results identified as “Positive.” The positive percent agreement (PPA) was defined as the ratio of the number of correctly predicted positives by the test method to the total number of positives identified by the reference method. The negative percent agreement (NPA) was defined as the ratio of the number of correctly predicted negatives by the test method to the total number of negatives identified by the reference method.

TABLE 3
Concordance table results for test method
vs current method from different IGH data

| Test vs | Training | | Training | | Test | | Test | | Test | |
|---|---|---|---|---|---|---|---|---|---|---|
| Current | data MMB | | data MMC | | data MMA | | Data MMB | | Data MMC | |
| method | Neg | Pos | Neg | Pos | Neg | Pos | Neg | Pos | Neg | Pos |
| Neg | 85 | 2 | 85 | 3 | 25 | 1 | 24 | 1 | 27 | 3 |
| Pos | 0 | 55 | 2 | 63 | 0 | 22 | 1 | 23 | 0 | 19 |

TABLE 4
Agreement results and corresponding 95% confidence intervals
for test method vs current method from different IGH data

| Test vs | | | | | | |
|---|---|---|---|---|---|---|
| Current | Training data MMB | | | Training data MMC | | |
| Method | mean | lower | upper | mean | lower | upper |
| PPA | 0.965 | 0.879 | 0.996 | 0.955 | 0.873 | 0.991 |
| NPA | 1 | 0.958 | 1 | 0.977 | 0.919 | 0.997 |

| Test vs | | | | | | | | | |
|---|---|---|---|---|---|---|---|---|---|
| Current | Test data MMA | | | Test data MMB | | | Training data MMC | | |
| Method | mean | lower | upper | mean | lower | upper | mean | lower | upper |
| PPA | 0.957 | 0.781 | 0.999 | 0.958 | 0.789 | 0.999 | 0.864 | 0.651 | 0.971 |
| NPA | 1 | 0.863 | 1 | 0.96 | 0.796 | 0.999 | 1 | 0.872 | 1 |

The results from Table 3 and Table 4 for various datasets indicated that the test method, which applied the algorithmic model, performed effectively for clonal prediction in comparison to results from the current method. For instance, in the training MMB dataset, 85 out of 85 negative results and 55 out of 57 positive results were correctly predicted by the test method relative to the current method results. The corresponding negative percent agreement (NPA) was 100% with a confidence interval of 0.958-1, and the positive percent agreement (PPA) was 0.965 with a confidence interval of 0.879-0.996. Similar high concordance was observed between the test method and current method results across other datasets.

In one embodiment, since IC IGH final results were based on the combined outcomes of MMA, MMB, and MMC, Table 5 and Table 6 provided the final concordance and agreement results of the test method versus the current method for IC IGH test samples. These tables also included comparison results of the test method versus the current NGS method.

From Tables 5 and 6, when comparing the test method with the current method, 29 out of 30 positive results and 23 out of 23 negative results were correctly predicted by the test method based on the current method results, resulting in a positive percent agreement (PPA) of 0.967 with a 95% confidence interval of 0.828-0.999 and a negative percent agreement (NPA) of 100% with a 95% confidence interval of 0.852-1. For the test method versus current NGS results, 28 out of 29 positive results and 23 out of 24 negative results were correctly predicted by the test method compared with current NGS results, yielding a PPA of 0.966 with a 95% confidence interval of 0.822-0.999 and an NPA of 0.958 with a 95% confidence interval of 0.789-0.999.

TABLE 5
Concordance table results for test method vs other method
result (from IGH test data for overall result)

| | | |
|---|---|---|
| Test Method | Test vs Current | Test vs NGS |
| vs Other | Result | Result |

| Method | Pos | Neg | Pos | Neg |
|---|---|---|---|---|
| Pos | 29 | 0 | 28 | 1 |
| Neg | 1 | 23 | 1 | 23 |

TABLE 6
Agreement results and corresponding 95% confidence
interval for test method vs other different methods
(from IGH test data for overall results)

Test Method

| vs Other | Test vs Current Result | | | Test vs NGS Result | | |
|---|---|---|---|---|---|---|
| Method | mean | lower | upper | mean | lower | upper |
| NPA | 1 | 0.852 | 1 | 0.958 | 0.789 | 0.999 |
| PPA | 0.967 | 0.828 | 0.999 | 0.966 | 0.822 | 0.999 |

In one embodiment, a similar method was applied to NGS data. Since NGS data output provided a larger dataset, data was grouped by V-J gene type based on information such as aligned sequence length, gene type (e.g., V-gene, J-gene), and percentage of total reads, as shown in Table 7. For each V-J gene type, the sum of the percentage of total reads was calculated as percentage reads, and the mean or median of the aligned sequence length was calculated as size. This yielded percentage read and size values for each V-J gene type, resulting in a data format similar to electropherogram data. The algorithmic method was then applied to these grouped data.

Given the larger data volume provided by NGS compared to electropherogram, which included more background information, different ranked subsets (10, 20, 30, and 40) of the data were tested, with the first ranked 20 subset providing superior results relative to other ranks. Thus, only the results from rank 20 are included, as shown in Tables 8 and 9.

TABLE 5
Example of paired NGS data

| | | Raw | V- | J- | % total | Cumulative |
|---|---|---|---|---|---|---|
| Rank | Length | count | gene | gene | reads | % |
| 1 | 147 | 3199 | Vg10 | JgP1 | 0.50775 | 0.50775 |
| 2 | 148 | 2049 | Vg10 | JgP2 | 0.32522 | 0.83297 |
| 4 | 112 | 1599 | Vg3 | Jg1/2 | 0.253795 | 1.355798 |
| 5 | 140 | 1518 | Vg3 | JgP2 | 0.240939 | 1.596737 |

In one embodiment, Table 8 provided the concordance results for the test method compared to various other methods for NGS training data. The test method applied the algorithmic model, using diagnosis results as a reference to develop the model. Table 9 included concordance results for the comparisons between the test method and diagnosis results, the test method and IC TRG method, and the test method and current NGS results. Table 9 presented the positive percent agreement (PPA) and negative percent agreement (NPA) results, along with the corresponding confidence intervals.

From Tables 8 and 9, it was observed that for the test method versus IC TRG method, 28 out of 30 negative results were correctly predicted by the test method based on IC TRG results, yielding an NPA of 0.933 with a 95% confidence interval of 0.779-0.992, and 28 out of 33 positive results were correctly predicted, resulting in a PPA of 0.848 with a 95% confidence interval of 0.681-0.949. For the test method versus current NGS method results, 27 out of 29 negative results were correctly predicted by the test method, with an NPA of 0.931 and a 95% confidence interval of 0.772-0.992, while 28 out of 33 positive results were correctly predicted, yielding a PPA of 0.848 with a 95% confidence interval of 0.681-0.949.

TABLE 6
Concordance table results for test method vs other
different methods (from NGS training data)

| Test Method | Test Result vs | | Test Results vs | | Test Result vs | |
|---|---|---|---|---|---|---|
| vs Other | Diagnosis Result | | TRG Result | | NGS Result | |
| Method | Neg | Pos | Neg | Pos | Neg | Pos |
| Neg | 27 | 6 | 28 | 5 | 27 | 5 |
| Pos | 2 | 30 | 2 | 28 | 2 | 28 |

TABLE 7
Agreement results and corresponding 95% confidence interval for
test method vs other different methods (from NGS training data)

| Test Method | Test Result vs | | | Test Result vs | | | Test Result vs | | |
|---|---|---|---|---|---|---|---|---|---|
| vs Other | Diagnosis Result | | | TRG result | | | NGS result | | |
| Method | mean | lower | upper | mean | lower | upper | mean | lower | upper |
| PPA | 0.833 | 0.672 | 0.936 | 0.848 | 0.681 | 0.949 | 0.848 | 0.681 | 0.949 |
| NPA | 0.931 | 0.772 | 0.992 | 0.933 | 0.779 | 0.992 | 0.931 | 0.772 | 0.992 |

### Example Embodiments

Embodiment 1. A computer-implemented method, comprising: receiving a detection-assay dataset comprising a set of detection values corresponding to measurements of nucleic acid fragments, the nucleic acid fragments generated from a biological sample of a subject, each detection count in the set corresponding to a nucleic-acid measurement; extracting one or more features of the detection-assay dataset, wherein at least one feature is extracted from a version of a detection value of a particular nucleic-acid measurement; inputting the one or more features of the detection-assay dataset into a model; and generating a computer-automated determination of homogeneity of the nucleic acid fragments in the biological sample of the subject based on an output of the model.

Embodiment 2. The computer-implemented method of embodiment 1, wherein extracting the one or more features of the detection-assay dataset comprises: selecting a representative detection value from the set of detection values in the detection-assay dataset according to one or more selection criteria; determining a version of the representative detection value; determining a detection-value-normalization reference based on aggregating one or more detection values in the set; and determining a normalized detection value of the representative detection value, wherein the one or more features of the detection-assay dataset comprise the normalized detection value.

Embodiment 3. The computer-implemented method of embodiment 2, wherein determining the detection value normalization reference based on aggregating one or more detection values in the set comprises: excluding one or more highest detection values in the set; and generating a sum of detection values in the set that excludes the one or more excluded detection values, wherein the sum of detection values is the detection-value-normalization reference.

Embodiment 4. The computer-implemented method of any of embodiments 1-3, wherein extracting the one or more features of the detection-assay dataset comprises: selecting a representative detection value from the detection values in the set according to one or more selection criteria; determining a nucleic-acid measurement of the representative detection value; classifying the representative detection value into a band according to the nucleic-acid measurement of the representative detection value; and determining an intensity of the representative detection value.

Embodiment 5. The computer-implemented method of any of embodiments 1-4, wherein extracting one or more features of the detection-assay dataset comprises: extracting one or more of the following: an area under curve of a detection value, a normalized value of a detection value, a number of detection values with intensities above a threshold, an aggregation of detection values of one or more detection values, a statistical determination of detection value of one or more detection values, a ratio of a detection value relative to a reference, and/or a modeling metric of one or more detection values relative to a distribution; and generating a feature vector representing the set of detection values in the detection-assay dataset, wherein the model is a machine learning model and the feature vector is used to input to the machine learning model.

Embodiment 6. The computer-implemented method of any of embodiments 1-5, wherein inputting the one or more features of the detection-assay dataset into the model comprises: identifying a detection value from which one of the features is extracted; determining a nucleic-acid measurement of the identified detection value; classifying the identified detection value into a band according to the nucleic-acid measurement of the identified detection value; applying a band-specific regression model selected from a set of regression models, the regression models in the set corresponding to a set of bands, wherein the applied band-specific regression model corresponds to the band to which the identified detection value is classified, and wherein the set of regression models is the model; and generating a score based on the band-specific regression model.

Embodiment 7. The computer-implemented method of any of embodiments 1-6, wherein the homogeneity is clonality.

Embodiment 8. The computer-implemented method of any of embodiments 1-7, wherein the model is selected from one or more of the following: a machine learning model, heuristic model, rule-based model, a regression model and/or a combination thereof.

Embodiment 9. The computer-implemented method of any of embodiments 1-8, wherein the model is a trained model and training of the model comprises: collecting training samples, the training samples generated from assays that apply one or more primers that target conserved genetic regions to biological samples; generating past detection-assay datasets from results of the assays; labeling the training samples based on homogeneity of the biological samples; and adjusting one or more parameters in the model based on the training samples, wherein the detection-assay dataset corresponding to the biological sample of the subject is generated from an assay that applies the one or more primers.

Embodiment 10. The computer-implemented method of any of embodiments 1-9, wherein the model is a trained model and training of the model comprises: initiating one or more parameters of the model; receiving training samples of past detection-assay datasets of homogeneous samples and non-homogeneous samples, the training samples associated with homogeneity labels; applying, in forward propagation, the model to predict homogeneity of one or more training samples; comparing predicted homogeneity to the homogeneity labels of the training samples; and adjusting, in backpropagation, the one or more parameters based on the comparison.

Embodiment 11. The computer-implemented method of any of embodiments 1-10, wherein generating the computer-automated determination of homogeneity of the nucleic acid fragments in the biological sample of the subject based on the output of the model comprises: receiving a score from the model; and comparing the score to a threshold to determine whether the biological sample of the subject is homogeneous or non-homogeneous.

Embodiment 12. The computer-implemented method of any of embodiments 1-11, wherein the detection-assay dataset is generated by a detection assay, the detection assay comprises: obtaining the biological sample of the subject; adding one or more primers to a solution of the biological sample, the one or more primers targeting conserved genetic regions; and performing a detection measurement of nucleic acids in the solution, wherein the detection measurement is a sequencing of the nucleic acids or an electropherogram that measures fragment sizes of the nucleic acids.

Embodiment 13. The computer-implemented method of any of embodiments 1-12, wherein detection-assay dataset is generated by sequencing, the sequencing comprises: obtaining the biological sample of the subject; adding one or more primers to a solution of the biological sample; and performing the sequencing to generate the sequence reads.

Embodiment 14. A computer product comprising one or more non-transitory computer-readable media configured to store code comprising instructions, wherein the instructions, when executed by one or more processors, cause the one or more processors to perform one or more steps recited in any of the preceding embodiments.

Embodiment 15. A system comprising: one or more processors; and memory configured to store code comprising instructions, wherein the instructions, when executed by the one or more processors, cause the one or more processors to perform one or more steps recited in any of the preceding embodiments.

Embodiment 16. A computer product comprising one or more non-transitory computer-readable media configured to store code comprising instructions, wherein the instructions, when executed by one or more processors, cause the one or more processors to: receive a detection dataset comprising a set of detection values corresponding to measurements of target entities, the target entities generated from a sample in a detection analysis, each detection value in the set corresponding to a measurement of one or more target entities; extract one or more features of the detection dataset, wherein at least one feature is extracted from a version of a particular detection value; input the one or more features of the detection dataset into a model; and generate a computer-automated determination of homogeneity of the target entities in the sample based on an output of the model.

Embodiment 17. The computer product of embodiment 16, wherein the instructions to extract the one or more features of the detection dataset comprises instructions to: select a representative detection value from the detection values in the detection dataset according to one or more selection criteria; determine a version of the representative detection value; determine a detection-value-normalization reference based on aggregating one or more detection values in the set; and determine a normalized detection value of the representative detection value, wherein the one or more features of the detection dataset comprise the normalized detection value.

Embodiment 18. The computer product of embodiment 17, wherein the instructions to determine the detection value normalization reference based on aggregating one or more detection values in the set comprises instructions to: exclude one or more highest detection values in the set; and generate a sum of detection values in the set that excludes the one or more excluded detection values, wherein the sum of detection values is the detection-value-normalization reference.

Embodiment 19. The computer product of embodiment 16, wherein the instruction to extract the one or more features of the detection dataset comprises instructions to: select a representative detection value from the detection values in the detection dataset according to one or more selection criteria; determine a measurement of a target entity of the representative detection value; classify the representative detection value into a band according to the measurement of a target entity of the representative detection value; and determine a version of the representative detection value.

Embodiment 20. The computer product of embodiment 16, wherein the instruction to extract the one or more features of the detection dataset comprises instructions to: extract one or more of the following: an area under curve of a detection value, a normalized value of a detection value, a number of detection values with intensities above a threshold, an aggregation of detection values of one or more detection values, a statistical determination of detection value of one or more detection values, a ratio of a detection value relative to a reference, and/or a modeling metric of one or more detection values relative to a distribution; and generate a feature vector representing the set of detection values in the detection dataset, wherein the model is a machine learning model and the feature vector is used to input to the machine learning model.

Embodiment 21. The computer product of embodiment 16, wherein the instruction to input the one or more features of the detection dataset into the model comprises instructions to: identify a detection value from which one of the features is extracted; determine a measurement of a target entity of the identified detection value; classify the identified detection value into a band according to the measurement of a target entity of the identified detection value; apply a band-specific regression model selected from a set of regression models, the regression models in the set corresponding to a set of bands, wherein the applied band-specific regression model corresponds to the band to which the identified detection value is classified, and wherein the set of regression models is the model; and generate a score based on the band-specific regression model.

Embodiment 22. The computer product of embodiment 21, wherein at least two of the regression models in the set include offset values that are offset from each other.

Embodiment 23. The computer product of embodiment 16, wherein the model is selected from one or more of the following: a machine learning model, heuristic model, rule-based model, a regression model and/or a combination thereof.

Embodiment 24. The computer product of embodiment 16, wherein the model is a trained model and training of the model comprises: collecting training samples from analyses that apply one or more probes that target specific entities; generating past detection datasets from the analyses; labeling the training samples based on homogeneity of the training samples; and adjusting one or more parameters in the model based on the training samples, wherein the detection dataset corresponding to the sample is generated from an analysis that applies the one or more probes.

Embodiment 25. The computer product of embodiment 16, wherein the model is a trained model and training of the model comprises: initiating one or more parameters of the model; receiving training samples of past detection datasets of homogeneous samples and non-homogeneous samples, the training samples associated with homogeneity labels; applying, in forward propagation, the model to predict homogeneity of one or more training samples; comparing predicted homogeneity to the homogeneity labels of the training samples; and adjusting, in backpropagation, the one or more parameters based on the comparison.

Embodiment 26. The computer product of embodiment 16, wherein the instructions to generate the computer-automated determination of the homogeneity of the sample fragments in the sample based on the output of the model comprises instructions to: receive a score from the model; and compare the score to a threshold to determine whether the sample is homogeneous or non-homogeneous.

Embodiment 27. The computer product of embodiment 16, wherein the detection dataset is generated by a test, the test comprises: obtaining the sample of a subject; adding one or more probes to the sample, the one or more probes targeting specific entities; and performing a detection measurement of target entities in the sample.

Embodiment 28. A system comprising: one or more processors; and memory configured to store code comprising instructions, wherein the instructions, when executed, cause the one or more processors to: receive a detection dataset comprising a set of detection values corresponding to measurements of target entities, the target entities generated from a sample in a detection analysis, each detection value in the set corresponding to a measurement of one or more target entities; extract one or more features of the detection dataset, wherein at least one feature is extracted from a version of a particular detection value; input the one or more features of the detection dataset into a model; and generate a computer-automated determination of homogeneity of the target entities in the sample based on an output of the model.

Embodiment 29. The system of embodiment 28, wherein the detection dataset is generated by the detection analysis carried out using a detection analysis kit, the detection analysis comprises: obtaining the sample of a subject; adding one or more probes to the sample, the one or more probes targeting specific entities; and performing a detection measurement of target entities in the sample.

Embodiment 30. The system of embodiment 28, wherein the instructions to extract the one or more features of the detection dataset comprises instructions to: select a representative detection value from the detection values in the detection dataset according to one or more selection criteria; determine a version of the representative detection value; determine a detection-value-normalization reference based on aggregating one or more detection values in the set; and determine a normalized detection value of the representative detection value, wherein the one or more features of the detection dataset comprise the normalized detection value.

Embodiment 31. The system of embodiment 30, wherein the instructions to determine the detection value normalization reference based on aggregating one or more detection values in the set comprises instructions to: exclude one or more highest detection values in the set; and generate a sum of detection values in the set that excludes the one or more excluded detection values, wherein the sum of detection values is the detection-value-normalization reference.

Embodiment 32. The system of embodiment 28, wherein the instruction to extract the one or more features of the detection dataset comprises instructions to: select a representative detection value from the detection values in the detection dataset according to one or more selection criteria; determine a measurement of a target entity of the representative detection value; classify the representative detection value into a band according to the measurement of a target entity of the representative detection value; and determine a version of the representative detection value.

Embodiment 33. A computer-implemented method, comprising: receiving a detection dataset comprising a set of detection values corresponding to measurements of target entities, the target entities generated from a sample in a detection analysis, each detection value in the set corresponding to a measurement of one or more target entities; extracting one or more features of the detection dataset, wherein at least one feature is extracted from a version of a particular detection value; inputting the one or more features of the detection dataset into a model; and generating a computer-automated determination of homogeneity of the target entities in the sample based on an output of the model.

Embodiment 34. The computer-implemented method of embodiment 33, wherein extracting the one or more features of the detection dataset comprises: selecting a representative detection value from the detection values in the detection dataset according to one or more selection criteria; determining a version of the representative detection value; determining a detection-value-normalization reference based on aggregating one or more detection values in the set; and determining a normalized detection value of the representative detection value, wherein the one or more features of the detection dataset comprise the normalized detection value.

Embodiment 35. The computer-implemented method of embodiment 33, wherein extracting the one or more features of the detection dataset comprises: selecting a representative detection value from the detection values in the detection dataset according to one or more selection criteria; determining a measurement of a target entity of the representative detection value; classifying the representative detection value into a band according to the measurement of a target entity of the representative detection value; and determining a version of the representative detection value.

Embodiment 36. A computer product comprising one or more non-transitory computer-readable media configured to store code comprising instructions, wherein the instructions, when executed by one or more processors, cause the one or more processors to: receive an electropherogram dataset comprising a set of peaks corresponding to measurements of amplicons, the amplicons amplified from nucleotide samples of a biological sample of a subject, each peak in the set corresponding to a fragment size and an intensity; extract one or more features of the peaks in the electropherogram dataset, wherein at least one feature is extracted from the intensity of a particular peak; input the one or more features of the peaks into a model that is trained based on training samples of past electropherogram datasets of homogeneous samples and non-homogeneous samples; and generate a computer-automated determination of homogeneity of the biological sample of the subject based on an output of the model.

Embodiment 37. The computer product of embodiment 36, wherein the instructions to extract the one or more features of the peaks in the electropherogram dataset comprises instructions to: select a representative peak from the peaks in the electropherogram dataset according to one or more selection criteria; determine an intensity of the representative peak; determine a peak normalization reference based on aggregating one or more peaks in the set; and determine a normalized peak intensity of the representative peak, wherein the one or more features of the peaks comprise the normalized peak intensity.

Embodiment 38. The computer product of embodiment 37, wherein the instructions to determine the peak normalization reference based on aggregating one or more peaks in the set comprises instructions to: exclude one or more highest peaks in the set; and generate a sum of peak intensities in the set excluding one or more excluded peaks, wherein the sum of peak intensities is the peak normalization reference.

Embodiment 39. The computer product of any of embodiments 36-38, wherein the instruction to extract the one or more features of the peaks in the electropherogram dataset comprises instructions to: select a representative peak from the peaks in the electropherogram dataset according to one or more selection criteria; determine a fragment size of the representative peak; classify the representative peak into a band according to the fragment size of the representative peak; and determine an intensity of the representative peak.

Embodiment 40. The computer product of any of embodiments 36-39, wherein the instruction to extract the one or more features of the peaks in the electropherogram dataset comprises instructions to: extract one or more of the following: an area under curve (AUC) of a peak, an intensity value of a peak, a normalized intensity value of a peak, a number of peaks with intensities above a threshold, an aggregation of peak intensities of one or more peaks, a statistical determination of peak intensities of one or more peaks, an intensity ratio of a peak relative to a reference, and/or a modeling metric of one or more peaks relative to a distribution; and generate a feature vector representing the set of peaks in the electropherogram dataset, wherein the model is a machine learning model and the feature vector is used to input to the machine learning model.

Embodiment 41. The computer product of any of embodiments 36-40, wherein the instruction to input the one or more features of the peaks into the model comprises instructions to: identify a peak from which one of the features is extracted; determine a fragment size of the identified peak; classify the identified peak into a band according to the fragment size of the identified peak; apply a band-specific regression model selected from a set of regression models, the regression models in the set corresponding to a set of bands, wherein the applied band-specific regression model corresponds to the band to which the identified peak is classified, and wherein the set of regression models is the model; and generate a score based on the band-specific regression model.

Embodiment 42. The computer product of embodiment 41, wherein at least two of the regression models in the set include offset values that are offset from each other.

Embodiment 43. The computer product of any of embodiments 36-42, wherein the model is selected from one or more of the following: a machine learning model, heuristic model, rule-based model, a regression model and/or a combination thereof.

Embodiment 44. The computer product of any of embodiments 36-43, wherein training of the model comprises: collecting the training samples, the training samples generated from assays that apply one or more primers that target conserved genetic regions to biological samples; generating the past electropherogram datasets from results of the assays; labeling the training samples based on homogeneity of the biological samples; and adjusting one or more parameters in the model based on the training samples, wherein the electropherogram dataset corresponding to the biological sample of the subject is generated from an assay that applies the one or more primers.

Embodiment 45. The computer product of any of embodiments 36-44, wherein training of the model comprises: initiating one or more parameters of the model; receiving the training samples of the past electropherogram datasets of homogeneous samples and non-homogeneous samples, the training samples associated with homogeneity labels; applying, in forward propagation, the model to predict homogeneity of one or more training samples; comparing predicted homogeneity to the homogeneity labels of the training samples; and adjusting, in backpropagation, the one or more parameters based on the comparison.

Embodiment 46. The computer product of any of embodiments 36-45, wherein the instructions to generate the computer-automated homogeneity determination of the biological sample of the subject based on the output of the model comprises instructions to: receive a score from the model; and compare the score to a threshold to determine whether the biological sample of the subject is homogeneous or non-homogeneous.

Embodiment 47. The computer product of any of embodiments 36-46, wherein the electropherogram dataset is generated by an assay, the assay comprises: obtaining the biological sample of the subject; adding one or more primers to a solution of the biological sample, the one or more primers targeting conserved genetic regions; performing a polymerase amplification to generate the amplicons; and performing a capillary electrophoresis on an amplification result to generate the electropherogram dataset.

Embodiment 48. The computer product of any of embodiments 36-47, wherein the biological sample is one of: a lymphoid tissue, a solid tumor biopsy, a tissue biopsy, FFPE, blood, whole blood, plasma, serum, urine, cerebrospinal fluid, fecal, saliva, tears, pleural fluid, pericardial fluid, or peritoneal fluid.

Embodiment 49. The computer product of any of embodiments 36-47, wherein the homogeneity is clonality.

Embodiment 50. A system comprising: an assay kit comprising oligonucleotide primers targeting one or more target genetic regions, the oligonucleotide primers configured to hybridize with nucleotides of a biological sample in the target genetic regions to generate, through an amplification process, a set of amplicons with an expected fragment size range; and an analytic device configured to generate a computer-automated determination of homogeneity of the biological sample, the analytic device comprising one or more processors and memory, the memory configured to store code comprising instructions, the instructions, when executed, cause the one or more processors to: receive an electropherogram dataset comprising a set of peaks in the expected fragment size range, the set of peaks corresponding to measurements of the amplicons, each peak in the set corresponding to a fragment size and an intensity; extract one or more features of the peaks in the electropherogram dataset, wherein at least one feature is determined from the intensity of a particular peak; input the one or more features of the peaks into a model that is trained based on training samples of past electropherogram datasets of homogeneous samples and non-homogeneous samples; and generate the computer-automated homogeneity determination of the biological sample based on an output of the model.

Embodiment 51. The system of embodiment 50, wherein the electropherogram dataset is generated by an assay carried out using the assay kit, the assay comprises: obtaining a biological sample of a subject; adding the oligonucleotide primers to a solution of the biological sample; performing a polymerase amplification to generate the amplicons corresponding to the target genetic regions; and performing a capillary electrophoresis on an amplification result to generate the electropherogram dataset; or.

Embodiment 52. The system of any of embodiments 50-51, wherein the instructions to extract the one or more features of the peaks in the electropherogram dataset comprises instructions to: select a representative peak from the peaks in the electropherogram dataset according to one or more selection criteria; determine an intensity of the representative peak; determine a peak normalization reference based on aggregating one or more peaks in the set; and determine a normalized peak intensity of the representative peak, wherein the one or more features of the peaks comprise the normalized peak intensity.

Embodiment 53. The system of embodiment 52, wherein the instructions to determine the peak normalization reference based on aggregating one or more peaks in the set comprises instructions to: exclude one or more highest in the set; and generate a sum of peak intensities in the set excluding one or more excluded peaks, wherein the sum of peak intensities is the peak normalization reference.

Embodiment 54. The system of any of embodiments 50-53, wherein the instruction to extract the one or more features of the peaks in the electropherogram dataset comprises instructions to: select a representative peak from the peaks in the electropherogram dataset according to one or more selection criteria; determine a fragment size of the representative peak; classify the representative peak into a band according to the fragment size of the representative peak; and determine an intensity of the representative peak.

Embodiment 55. The system of any of embodiments 50-54, wherein the one or more target genetic regions are located in one or more of the following genes: ACVR1B, AKT3, AMER1, APC, ARID1A, ARID1B, ARID2, ASXL1, ASXL2, ATM, ATR, BAP1 BCL2, BCL6, BCORL1, BCR, BLM, BRAF, BRCA1, BTG1, CASP8, CBL, CCND3, CCNE1, CD74, CDC73, CDK12, CDKN2A, CHD2, CJD2, CREBBP, CSF1R, CTCF, CTNNB1, DICER1, DNAJB1, DNMT1, DNMT3A, DNMT3B, DOT1L, EED, EGFR, EIF1AX, EP300, EPHA3, EPHA5, EPHB1, ERBB2, ERBB4, ERCC2, ERCC3, ERCC4, ESR1, FAM46C, FANCA, FANCC, FANCD2, FANCE, FAT1, FBXW7, FGFR3, FLCN, FLT1, FLT3 FOXO1, FUBP1, FYN, GATA3, GPR124, GRIN2A, GRM3, H3F3A, HIST1H1C, IDH1, IDH2, IKZF1, IL7R, INPP4B, IRF4, IRS1, IRS2, JAK2, KAT6A, KDM6A, KEAP1, KIF5B, KIT, KLF4, KLH6, KMT2C, KRAS, LMAP1, LRP1B, LZTR1, MAP3K1, MCL1, MGA, MSH2, MSH6, MSTIR, MTOR, MYD88, NPM1, NRAS, NTRK1, NTRK2, NUP93, NUTM1, PAX3, PAX8, PBRM1, PGR, PHOX2B, PIK3CA, POLE, PTCH1, PTEN, PTPN11, PTPRT, RAD21, RAF1, RANBP2, RB1, REL, RFWD2, RHOA, RPTOR, RUNX1, RUNX1T1, SDHA, SHQ1, SLIT2, SMAD4, SMARCA4, SMARCD1, SNCAIP, SOCS1, SPEN, SPTA1, SUZ12, TET1, TET2, TGFBR, TNFRSF14, and/or clonality detection in immunoglobulin gene rearrangements (IGH, IGK, TRB and/or TRG).

Embodiment 56. A computer product comprising one or more non-transitory computer-readable media configured to store code comprising instructions, wherein the instructions, when executed by one or more processors, cause the one or more processors to: receive a sequencing dataset comprising a set of read counts corresponding to measurement of sequence reads, the sequence reads being nucleotide sequences determined from a biological sample of a subject, each read count in the set corresponding to a sequence read; extract one or more features of the sequencing dataset, wherein at least one feature is extracted from a version of the read count of a particular sequence read; input the one or more features of the sequencing dataset into a model; and generate a computer-automated determination of homogeneity of the nucleotide sequences in the biological sample of the subject based on an output of the model.

Embodiment 57. The computer product of embodiment 56, wherein the instructions to extract the one or more features of the sequencing dataset comprises instructions to: select a representative read count from the set of read counts in the sequencing dataset according to one or more selection criteria; determine a version of a value of the representative read count; determine a read-count-normalization reference based on aggregating one or more read counts in the set; and determine a normalized read count of the representative read count, wherein the one or more features of the sequencing dataset comprise the normalized read count.

Embodiment 58. The computer product of embodiment 57, wherein the instructions to determine the read count normalization reference based on aggregating one or more read counts in the set comprises instructions to: exclude one or more highest read counts in the set; and generate a sum of the read counts based on the read counts in the set excluding one or more excluded read counts, wherein the sum of the read counts is the read-count-normalization reference.

Embodiment 59. The computer product of any of embodiments 56-58, wherein the instructions to extract the one or more features of the sequencing dataset comprises instructions to: select a representative read count from the set of read counts in the sequencing dataset according to one or more selection criteria; determine a sequence read of the representative read count; classify the representative read count into a group according to the sequence read of the representative read count; and determine a version of a value of the representative read count.

Embodiment 60. The computer product of any of embodiments 56-59, wherein a version of the read count corresponds of one of the following: a raw value of the read count, a percentage value of the read count, a number of read counts above a threshold, an aggregation of one or more read counts, a statistical determination of the read count, a ratio of the read count relative to a reference, and/or a modeling metric of one or more read counts relative to a distribution, wherein the one or more features are inputted as a feature vector, and wherein the model is a machine learning model and the feature vector is used to input to the machine learning model.

Embodiment 61. The computer product of any of embodiments 56-60, wherein the instruction to input the one or more features of the sequencing dataset into the model comprises instructions to: identify a read count from which one of the features is extracted; determine a sequence read of the identified read count; apply a regression model; and generate a score based on the regression model.

Embodiment 62. The computer product of any of embodiments 56-61, wherein the model is selected from one or more of the following: a machine learning model, heuristic model, rule-based model, a regression model and/or a combination thereof.

Embodiment 63. The computer product of any of embodiments 56-62, wherein training of the model comprises: collecting the training samples, the training samples generated from sequencing runs that apply one or more primers that target conserved genetic regions to biological samples; generating the past sequencing datasets from results of the sequencing runs; labeling the training samples based on homogeneity of the biological samples; and adjusting one or more parameters in the model based on the training samples, wherein the sequencing dataset corresponding to the biological sample of the subject is generated from a sequencing run that applies the one or more primers.

Embodiment 64. The computer product of any of embodiments 56-63, wherein training of the model comprises: initiating one or more parameters of the model; receiving the training samples of the past sequencing datasets of homogeneity samples and non-homogeneity samples, the training samples associated with homogeneity labels; applying, in forward propagation, the model to predict homogeneity of one or more training samples; comparing predicted homogeneity to the homogeneity labels of the training samples; and adjusting, in backpropagation, the one or more parameters based on the comparison.

Embodiment 65. The computer product of any of embodiments 56-64, wherein the instructions to generate the computer-automated determination of homogeneity of the biological sample of the subject based on the output of the model comprises instructions to: receive a score from the model; and compare the score to a threshold to determine whether the biological sample of the subject is homogeneous or non-homogeneous.

Embodiment 66. The computer product of any of embodiments 56-65, wherein the sequencing dataset is generated by a sequencing run, the sequencing run comprises: obtaining the biological sample of the subject; adding one or more primers to a solution of the biological sample, the one or more primers targeting conserved genetic regions with T-cell receptor gamma chain gene; performing sequencing to generate the sequencing dataset.

Embodiment 67. The computer product of any of embodiments 56-66, wherein the biological sample is one of: a lymphoid tissue, a solid tumor biopsy, a tissue biopsy, FFPE, blood, whole blood, plasma, serum, urine, cerebrospinal fluid, fecal, saliva, tears, pleural fluid, pericardial fluid, or peritoneal fluid.

Embodiment 68. The computer product of any of embodiments 56-67, wherein the sequencing dataset is generated by a massively parallel sequencing.

Embodiment 69. The computer product of any of embodiments 55-68, wherein the homogeneity is clonality.

### ADDITIONAL CONSIDERATION

Current on-market T-Cell Receptor Gamma Gene Rearrangement Assay requires a visual review of electropherogram results to determine clonality status. It is subjective, not reproducible and time consuming.

The new analysis method is objective and does not require visual inspection of electropherogram results. Since the method is based on objective analysis of the electropherogram peak output profile of the test result, it is possible for the assay analysis to be automated.

The analysis method is based on objective analysis of electropherogram results and provides good prediction results. It also does not need a visual review of electropherogram results.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcodes, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or distributively, comprise instructions that, when executed by one or more processors, cause the one or more processors to perform, individually or distributively, the steps of the instructions stored on the one or more computer-readable media. Similarly, a processor comprises one or more processors or processing units that, individually or distributively, perform the steps of instructions stored on a computer-readable medium.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer-readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process, where the information is stored on a non-transitory, tangible computer-readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limited, of the scope of the invention, which is set forth in the following claims.

1. A computer product comprising one or more non-transitory computer-readable media configured to store code comprising instructions, wherein the instructions, when executed by one or more processors, cause the one or more processors to:
receive a detection dataset comprising a set of detection values corresponding to measurements of target entities, the target entities generated from a sample in a detection analysis, each detection value in the set corresponding to a measurement of one or more target entities;
extract one or more features of the detection dataset, wherein at least one feature is extracted from a version of a particular detection value;
input the one or more features of the detection dataset into a model; and
generate a computer-automated determination of homogeneity of the target entities in the sample based on an output of the model.

2. The computer product of claim 1, wherein the instructions to extract the one or more features of the detection dataset comprises instructions to:
select a representative detection value from the detection values in the detection dataset according to one or more selection criteria;
determine a version of the representative detection value;
determine a detection-value-normalization reference based on aggregating one or more detection values in the set; and
determine a normalized detection value of the representative detection value, wherein the one or more features of the detection dataset comprise the normalized detection value.

3. The computer product of claim 2, wherein the instructions to determine the detection value normalization reference based on aggregating one or more detection values in the set comprises instructions to:
exclude one or more highest detection values in the set; and
generate a sum of detection values in the set that excludes the one or more excluded detection values, wherein the sum of detection values is the detection-value-normalization reference.

4. The computer product of claim 1, wherein the instruction to extract the one or more features of the detection dataset comprises instructions to:
select a representative detection value from the detection values in the detection dataset according to one or more selection criteria;
determine a measurement of a target entity of the representative detection value;
classify the representative detection value into a band according to the measurement of a target entity of the representative detection value; and
determine a version of the representative detection value.

5. The computer product of claim 1, wherein the instruction to extract the one or more features of the detection dataset comprises instructions to:
extract one or more of the following: an area under curve of a detection value, a normalized value of a detection value, a number of detection values with intensities above a threshold, an aggregation of detection values of one or more detection values, a statistical determination of detection value of one or more detection values, a ratio of a detection value relative to a reference, and/or a modeling metric of one or more detection values relative to a distribution; and
generate a feature vector representing the set of detection values in the detection dataset, wherein the model is a machine learning model and the feature vector is used to input to the machine learning model.

6. The computer product of claim 1, wherein the instruction to input the one or more features of the detection dataset into the model comprises instructions to:
identify a detection value from which one of the features is extracted;
determine a measurement of a target entity of the identified detection value;
classify the identified detection value into a band according to the measurement of a target entity of the identified detection value;
apply a band-specific regression model selected from a set of regression models, the regression models in the set corresponding to a set of bands, wherein the applied band-specific regression model corresponds to the band to which the identified detection value is classified, and wherein the set of regression models is the model; and
generate a score based on the band-specific regression model.

7. The computer product of claim 6, wherein at least two of the regression models in the set include offset values that are offset from each other.

8. The computer product of claim 1, wherein the model is selected from one or more of the following: a machine learning model, heuristic model, rule-based model, a regression model and/or a combination thereof.

9. The computer product of claim 1, wherein the model is a trained model and training of the model comprises:
collecting training samples from analyses that apply one or more probes that target specific entities;
generating past detection datasets from the analyses;
labeling the training samples based on homogeneity of the training samples; and
adjusting one or more parameters in the model based on the training samples, wherein the detection dataset corresponding to the sample is generated from an analysis that applies the one or more probes.

10. The computer product of claim 1, wherein the model is a trained model and training of the model comprises:
initiating one or more parameters of the model;
receiving training samples of past detection datasets of homogeneous samples and non-homogeneous samples, the training samples associated with homogeneity labels;
applying, in forward propagation, the model to predict homogeneity of one or more training samples;
comparing predicted homogeneity to the homogeneity labels of the training samples; and
adjusting, in backpropagation, the one or more parameters based on the comparison.

11. The computer product of claim 1, wherein the instructions to generate the computer-automated determination of the homogeneity of the sample fragments in the sample based on the output of the model comprises instructions to:
receive a score from the model; and
compare the score to a threshold to determine whether the sample is homogeneous or non-homogeneous.

12. The computer product of claim 1, wherein the detection dataset is generated by a test, the test comprises:
obtaining the sample of a subject;
adding one or more probes to the sample, the one or more probes targeting specific entities; and
performing a detection measurement of target entities in the sample.

13. A system comprising:
one or more processors; and
memory configured to store code comprising instructions, wherein the instructions, when executed, cause the one or more processors to:
receive a detection dataset comprising a set of detection values corresponding to measurements of target entities, the target entities generated from a sample in a detection analysis, each detection value in the set corresponding to a measurement of one or more target entities;
extract one or more features of the detection dataset, wherein at least one feature is extracted from a version of a particular detection value;
input the one or more features of the detection dataset into a model; and
generate a computer-automated determination of homogeneity of the target entities in the sample based on an output of the model.

14. The system of claim 13, wherein the detection dataset is generated by the detection analysis carried out using a detection analysis kit, the detection analysis comprises:
obtaining the sample of a subject;
adding one or more probes to the sample, the one or more probes targeting specific entities; and
performing a detection measurement of target entities in the sample.

15. The system of claim 13, wherein the instructions to extract the one or more features of the detection dataset comprises instructions to:
select a representative detection value from the detection values in the detection dataset according to one or more selection criteria;
determine a version of the representative detection value;
determine a detection-value-normalization reference based on aggregating one or more detection values in the set; and
determine a normalized detection value of the representative detection value, wherein the one or more features of the detection dataset comprise the normalized detection value.

16. The system of claim 15, wherein the instructions to determine the detection value normalization reference based on aggregating one or more detection values in the set comprises instructions to:
exclude one or more highest detection values in the set; and
generate a sum of detection values in the set that excludes the one or more excluded detection values, wherein the sum of detection values is the detection-value-normalization reference.

17. The system of claim 13, wherein the instruction to extract the one or more features of the detection dataset comprises instructions to:
select a representative detection value from the detection values in the detection dataset according to one or more selection criteria;
determine a measurement of a target entity of the representative detection value;
classify the representative detection value into a band according to the measurement of a target entity of the representative detection value; and
determine a version of the representative detection value.

18. A computer-implemented method, comprising:
receiving a detection dataset comprising a set of detection values corresponding to measurements of target entities, the target entities generated from a sample in a detection analysis, each detection value in the set corresponding to a measurement of one or more target entities;
extracting one or more features of the detection dataset, wherein at least one feature is extracted from a version of a particular detection value;
inputting the one or more features of the detection dataset into a model; and
generating a computer-automated determination of homogeneity of the target entities in the sample based on an output of the model.

19. The computer-implemented method of claim 18, wherein extracting the one or more features of the detection dataset comprises:
selecting a representative detection value from the detection values in the detection dataset according to one or more selection criteria;
determining a version of the representative detection value;
determining a detection-value-normalization reference based on aggregating one or more detection values in the set; and
determining a normalized detection value of the representative detection value, wherein the one or more features of the detection dataset comprise the normalized detection value.

20. The computer-implemented method of claim 18, wherein extracting the one or more features of the detection dataset comprises:
selecting a representative detection value from the detection values in the detection dataset according to one or more selection criteria;
determining a measurement of a target entity of the representative detection value;
classifying the representative detection value into a band according to the measurement of a target entity of the representative detection value; and
determining a version of the representative detection value.
